(19) United States
(12) Patent Application Publication
(10) Pub. No.: US 2025/0114088 A1
(43) Pub. Date: Apr. 10, 2025

(54) SUTURE LOCK ASSEMBLY WITH A SUTURE TENSIONER

(71) Applicant: EDWARDS LIFESCIENCES CORPORATION, Irvine, CA (US)

(72) Inventors: Kurt Kelly Reed, Costa Mesa, CA (US); Tri D. Tran, Fountain Valley, CA (US)

(21) Appl. No.: 18/984,170

(22) Filed: Dec. 17, 2024

Related U.S. Application Data
(63) Continuation of application No. PCT/US2023/025726, filed on Jun. 20, 2023.
(60) Provisional application No. 63/366,731, filed on Jun. 21, 2022.

(51) Int. Cl. A61B 17/04

(52) U.S. Cl. CPC A61B 17/0469; A61B 17/0487; A61B 2017/0488; A61B 2017/0496

(57) ABSTRACT

Suture lock assemblies with suture tensioners are disclosed herein. A suture lock assembly can comprise a suture tensioner configured to be selectively rotated to collect a length of a suture. The suture tensioner comprises a pair of spaced-apart posts and a connecting body coupled to each of the posts. A suture lock assembly also can comprise a suture tensioner, a tensioner housing, and a quick release suture mechanism. The tensioner housing at least partially receives the suture tensioner and includes a suture entry port and a suture exit port. The suture tensioner is configured to be selectively rotated relative to the tensioner housing to selectively increase a length of a suture that is enclosed by the tensioner housing. The quick release suture mechanism comprises a quick release dock port and a suture anchor cap configured to be selectively coupled to and uncoupled from the quick release dock port.

### CROSS REFERENCE TO RELATED APPLICATION

This application is a continuation of PCT Patent Application No. PCT/US2023/025648, filed on Jun. 19, 2023, which claims the benefit of U.S. Provisional Application No. 63/366,731, filed Jun. 21, 2022, each of these applications being incorporated by reference herein in its entirety.

### FIELD

The present disclosure relates to a suture lock assembly with a suture tensioner, such as may be utilized in conjunction with a delivery apparatus for delivering a prosthetic implant into a patient's body.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (e.g., stents) and artificial valves, as well as a number of known methods of implanting these devices and valves in humans. Percutaneous and minimally-invasive surgical approaches are used in various procedures to deliver prosthetic medical devices to locations inside the body that are not readily accessible by surgery or where access without surgery is desirable. In one specific example, a prosthetic heart valve can be mounted in a crimped state on the distal end of a delivery apparatus and advanced through the patient's vasculature (e.g., through a femoral artery and the aorta) until the prosthetic heart valve reaches the implantation site in the heart. The prosthetic heart valve is then expanded to its functional size, for example, by inflating a balloon on which the prosthetic valve is mounted, actuating a mechanical actuator that applies an expansion force to the prosthetic heart valve, or by deploying the prosthetic heart valve from a sheath of the delivery apparatus so that the prosthetic heart valve can self-expand to its functional size.

### SUMMARY

Described herein are delivery apparatuses and methods for implanting prosthetic heart valves. The disclosed delivery apparatuses and methods can, for example, provide improved releasable suture connections to prosthetic heart valves and/or to docking devices for anchoring the prosthetic heart valves. As such, the devices and methods disclosed herein can, among other things, overcome one or more of the deficiencies of typical prosthetic heart valve delivery apparatuses.

A delivery apparatus for a prosthetic implant can comprise a handle and one or more shafts coupled to the handle.

In some examples, the one or more shafts comprises a delivery shaft configured to be advanced through a patient's vasculature to an implantation site and configured to retain an implantable device.

In some examples, the implantable device comprises a docking device.

In some examples, the delivery apparatus comprises a pusher assembly configured to deploy and/or implant the implantable device at the implantation site.

In some examples, the implantable device is coupled to the delivery apparatus via a suture that can be cut for removal of the implantable device from the delivery apparatus.

In some examples, the delivery apparatus comprises a suture lock assembly that is configured to engage the suture.

In some examples, the suture lock assembly comprises a suture tensioner configured to engage the suture.

In some examples, the suture tensioner is configured to selectively increase or decrease a tension of the suture.

In some examples, the suture tensioner is configured to be selectively rotated about a tensioner central axis to collect a length of the suture.

In some examples, the suture tensioner comprises a pair of spaced-apart posts, each post configured to revolve around the tensioner central axis as the suture tensioner rotates.

In some examples, the suture tensioner is configured to rotate to transition the suture tensioner among a plurality of tensioner configurations defined between and comprising a released configuration, in which the posts are positioned to exert no force on the suture, and a fully tensioned configuration, in which the posts are positioned to engage the suture to yield a maximum tension in the suture when the suture is connected to the implantable device.

In some examples, the suture lock assembly comprises a tensioner housing that at least partially receives the suture tensioner.

In some examples, the suture lock assembly comprises a quick release suture mechanism configured to be selectively transitioned between a locked configuration and an unlocked configuration, and the quick release suture mechanism is configured such that the suture may be removed from the suture lock assembly when the quick release suture mechanism is in the unlocked configuration.

In some examples, the quick release suture mechanism comprises a quick release dock port and a suture anchor cap configured to be selectively coupled to and uncoupled from the quick release dock port.

In one representative example, a suture lock assembly comprises a suture tensioner configured to engage a suture that is configured to be connected to an implantable device. The suture tensioner is configured to be selectively rotated about a tensioner central axis to collect a length of the suture. The suture tensioner comprises a pair of spaced-apart posts and a connecting body coupled to each of the posts. Each post is configured to revolve around the tensioner central axis as the suture tensioner rotates.

In another representative example, a suture lock assembly comprises a suture tensioner, a tensioner housing, and a quick release suture mechanism. The suture tensioner is configured to engage a suture that is configured to be connected to an implantable device. The tensioner housing at least partially receives the suture tensioner and includes a suture entry port and a suture exit port such that the suture extends through the tensioner housing between the suture entry port and the suture exit port. The quick release suture mechanism is coupled to the suture exit port. The suture tensioner is configured to be selectively rotated relative to the tensioner housing about a tensioner central axis to selectively increase a length of the suture that is enclosed by the tensioner housing. The suture tensioner comprises a pair of spaced-apart posts and a connecting body coupled to each of the posts. Each post is configured to revolve around the tensioner central axis as the suture tensioner rotates relative to the tensioner housing. The quick release suture mechanism comprises a quick release dock port and a suture anchor cap. The quick release dock port is configured to be attached to the suture exit port, and the suture anchor cap is configured to be selectively coupled to and uncoupled from the quick release dock port. The quick release suture mechanism is configured to be selectively transitioned between a locked configuration, in which the quick release suture mechanism maintains the suture in a fixed position relative to the suture exit port, and an unlocked configuration, in which the suture may be removed from the suture exit port. The quick release suture mechanism is in the locked configuration when the suture anchor cap is operatively coupled to the quick release dock port, and the quick release suture mechanism is in the unlocked configuration when the suture anchor cap is removed from the quick release dock port. The suture extends between and terminates at each of a suture anchored end and a suture free end. The suture anchor cap comprises a suture anchor location, and the suture anchor cap is configured to be operatively coupled to the suture anchored end at the suture anchor location such that the suture anchored end is restricted from being removed from the suture anchor cap during operative use of the quick release suture mechanism.

In some examples, a suture lock assembly comprises one or more of the components recited in examples 59-126 below.

The various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosure will become more apparent from the following detailed description, claims, and accompanying figures.

### BRIEF DESCRIPTION OF THE DRAWINGS

FIG. 1 schematically illustrates a first stage in an exemplary mitral valve replacement procedure where a guide catheter and a guidewire are inserted into a blood vessel of a patient and navigated through the blood vessel and into a heart of the patient, towards a native mitral valve of the heart.

FIG. 2A schematically illustrates a second stage in the exemplary mitral valve replacement procedure where a docking device delivery apparatus extending through the guide catheter is implanting a docking device for a prosthetic heart valve at the native mitral valve.

FIG. 2B schematically illustrates a third stage in the exemplary mitral valve replacement procedure where the docking device of FIG. 2A is fully implanted at the native mitral valve of the patient and the docking device delivery apparatus has been removed from the patient.

FIG. 3A schematically illustrates a fourth stage in the exemplary mitral valve replacement procedure where a prosthetic heart valve delivery apparatus extending through the guide catheter is implanting a prosthetic heart valve in the implanted docking device at the native mitral valve.

FIG. 3B schematically illustrates a fifth stage in the exemplary mitral valve replacement procedure where the prosthetic heart valve is fully implanted within the docking device at the native mitral valve and the prosthetic heart valve delivery apparatus has been removed from the patient.

FIG. 4 schematically illustrates a sixth stage in the exemplary mitral valve replacement procedure where the guide catheter and the guidewire have been removed from the patient.

FIG. 5 is a perspective view of a docking device, according to one example.

FIG. 6A is side view of a delivery apparatus for a docking device, according to one example.

FIG. 6B depicts a portion of the delivery apparatus of FIG. 6A.

FIG. 6C is a perspective view of a suture lock assembly of the delivery apparatus of FIGS. 6A-6B.

FIG. 6D is a side view of the suture lock assembly of FIG. 6C.

FIG. 7 schematically illustrates a suture lock assembly that comprises a spool and a quick release suture mechanism, according to various examples.

FIG. 8 is a cross-sectional side view of a quick release suture mechanism in a locked configuration, according to a first example.

FIG. 9 is a cross-sectional side view of a quick release suture mechanism in an unlocked configuration, according to a second example.

FIG. 10A depicts a quick release suture mechanism in a locked configuration, according to a third example.

FIG. 10B depicts the quick release suture mechanism of FIG. 10A in an unlocked configuration.

FIG. 10C depicts the quick release suture mechanism of FIGS. 10A-10B with a release suture beginning to be withdrawn from the quick release suture mechanism.

FIG. 10D depicts the quick release suture mechanism of FIGS. 10A-10B with the release suture fully withdrawn from the quick release suture mechanism.

FIG. 11 schematically illustrates a suture lock assembly that comprises a suture tensioner and a quick release suture mechanism, according to various examples.

FIG. 12A depicts a suture tensioner in a released configuration, according to one example.

FIG. 12B depicts the suture tensioner of FIG. 12A beginning to engage a release suture as the suture tensioner is rotated.

FIG. 12C depicts the suture tensioner of FIGS. 12A-12B engaging the release suture as the suture tensioner is rotated farther.

FIG. 12D depicts the suture tensioner of FIGS. 12A-12C engaging the release suture as the suture tensioner is rotated still farther.

FIG. 12E depicts the suture tensioner of FIGS. 12A-12D engaging the release suture in a fully tensioned configuration.

FIG. 12F depicts the suture tensioner of FIGS. 12A-12E with the suture tensioner rotated from the fully tensioned configuration toward the released configuration and with no distally directed force being exerted upon the release suture.

FIG. 12G depicts the suture tensioner of FIGS. 12A-12F with the suture tensioner rotated farther toward the released configuration and with no distally directed force being exerted upon the release suture.

FIG. 13 depicts a suture tensioner engaging a release suture, according to another example.

FIG. 14A is a cross-sectional side view of a suture lock assembly that comprises a suture tensioner and a quick release suture mechanism, according to a first example.

FIG. 14B is a top view of the suture lock assembly of FIG. 14A.

FIG. 15A is a top view of a suture lock assembly that comprises a suture tensioner and a quick release suture mechanism, according to a second example.

FIG. 15B is a front side perspective view of the suture lock assembly of FIG. 15A.

FIG. 15C is a rear side perspective view of the suture lock assembly of FIGS. 15A-15B.

FIG. 15D is a cross-sectional view of the suture lock assembly of FIGS. 15A-15C.

FIG. 15E is another cross-sectional view of the suture lock assembly of FIGS. 15A-15D as viewed along the line 15E-15E of FIG. 15A.

FIG. 15F is another cross-sectional view of the suture lock assembly of FIGS. 15A-15E as viewed along the line 15F-15F of FIG. 15B.

FIG. 15G is an exploded view of the suture lock assembly of FIGS. 15A-15E.

FIG. 15H depicts a suture tensioner of the suture lock assembly of FIGS. 15A-15G.

FIG. 15I is a cross-sectional view of a suture anchor cap of the suture lock assembly of FIGS. 15A-15H as viewed along the line 15I-15I of FIG. 15G.

FIG. 15J is a cross-sectional view of the suture anchor cap of FIG. 15I operatively coupled to the quick release suture mechanism of the suture lock assembly of FIGS. 15A-15I and operatively coupled to a release suture, according to a first example.

FIG. 15K is a cross-sectional view of the suture anchor cap of FIGS. 15I-15J operatively coupled to the quick release suture mechanism of the suture lock assembly of FIGS. 15A-15J and operatively coupled to a release suture, according to a second example.

FIG. 15L is a cross-sectional view of the suture anchor cap of FIGS. 15I-15K operatively coupled to the quick release suture mechanism of the suture lock assembly of FIGS. 15A-15K and operatively coupled to a release suture, according to a third example.

FIG. 15M is a cross-sectional view of the suture anchor cap of FIGS. 15I-15L operatively coupled to the quick release suture mechanism of the suture lock assembly of FIGS. 15A-15L and operatively coupled to a release suture, according to a fourth example.

### DETAILED DESCRIPTION

### General Considerations

It should be understood that the disclosed examples can be adapted to deliver and implant prosthetic devices in any of the native annuluses of the heart (e.g., the pulmonary, mitral, and tricuspid annuluses), and can be used with any of various delivery approaches (e.g., retrograde, antegrade, transseptal, transventricular, transatrial, etc.).

For purposes of this description, certain aspects, advantages, and novel features of the examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present or problems be solved. The technologies from any example can be combined with the technologies described in any one or more of the other examples. In view of the many possible examples to which the principles of the disclosed technology may be applied, it should be recognized that the illustrated examples are only preferred examples and should not be taken as limiting the scope of the disclosed technology.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the term “includes” means “comprises.” Further, the terms “coupled” and “connected” generally mean electrically, electromagnetically, and/or physically (e.g., mechanically or chemically) coupled or linked and does not exclude the presence of intermediate elements between the coupled or associated items absent specific contrary language.

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device away from the implantation site and toward the user (e.g., out of the patient's body), while distal motion of the device is motion of the device away from the user and toward the implantation site (e.g., into the patient's body). The terms “longitudinal” and “axial” refer to an axis extending in the proximal and distal directions, unless otherwise expressly defined.

As used herein, the term “approximately” and “about” means the listed value and any value that is within 10% of the listed value. For example, “about 1 mm” means any value between about 0.9 mm and about 1.1 mm, inclusive.

As used herein, “e.g.” means “for example,” and “i.e.” means “that is.”

Directions and other relative references (e.g., inner, outer, upper, lower, etc.) may be used to facilitate discussion of the drawings and principles herein, but are not intended to be limiting. For example, certain terms may be used such as “inside,” “outside,” “top,” “down,” “interior,” “exterior,” and the like. Such terms are used, where applicable, to provide some clarity of description when dealing with relative relationships, particularly with respect to the illustrated examples. Such terms are not, however, intended to imply absolute relationships, positions, and/or orientations. For example, with respect to an object, an “upper” part can become a “lower” part simply by turning the object over. Nevertheless, it is still the same part and the object remains the same. As used herein, “and/or” means “and” or “or,” as well as “and” and “or.”

### Delivery Techniques

For implanting a prosthetic valve within the native aortic valve via a transfemoral delivery approach, the prosthetic valve is mounted in a radially compressed state along the distal end portion of a delivery apparatus. The prosthetic valve and the distal end portion of the delivery apparatus are inserted into a femoral artery and are advanced into and through the descending aorta, around the aortic arch, and through the ascending aorta. The prosthetic valve is positioned within the native aortic valve and radially expanded (e.g., by inflating a balloon, actuating one or more actuators of the delivery apparatus, or deploying the prosthetic valve from a sheath to allow the prosthetic valve to self-expand). Alternatively, a prosthetic valve can be implanted within the native aortic valve in a transapical procedure, whereby the prosthetic valve (on the distal end portion of the delivery apparatus) is introduced into the left ventricle through a surgical opening in the chest and the apex of the heart and the prosthetic valve is positioned within the native aortic valve. Alternatively, in a transaortic procedure, a prosthetic valve (on the distal end portion of the delivery apparatus) is introduced into the aorta through a surgical incision in the ascending aorta, such as through a partial J-sternotomy or right parasternal mini-thoracotomy, and then advanced through the ascending aorta toward the native aortic valve.

For implanting a prosthetic valve within the native mitral valve via a transseptal delivery approach, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, into the right atrium, across the atrial septum (through a puncture made in the atrial septum), into the left atrium, and toward the native mitral valve. Alternatively, a prosthetic valve can be implanted within the native mitral valve in a transapical procedure, whereby the prosthetic valve (on the distal end portion of the delivery apparatus) is introduced into the left ventricle through a surgical opening in the chest and the apex of the heart and the prosthetic valve is positioned within the native mitral valve.

For implanting a prosthetic valve within the native tricuspid valve, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, and into the right atrium, and the prosthetic valve is positioned within the native tricuspid valve. A similar approach can be used for implanting the prosthetic valve within the native pulmonary valve or the pulmonary artery, except that the prosthetic valve is advanced through the native tricuspid valve into the right ventricle and toward the pulmonary valve/pulmonary artery.

Another delivery approach is a transatrial approach whereby a prosthetic valve (on the distal end portion of the delivery apparatus) is inserted through an incision in the chest and an incision made through an atrial wall (of the right or left atrium) for accessing any of the native heart valves. Atrial delivery can also be made intravascularly, such as from a pulmonary vein. Still another delivery approach is a transventricular approach whereby a prosthetic valve (on the distal end portion of the delivery apparatus) is inserted through an incision in the chest and an incision made through the wall of the right ventricle (typically at or near the base of the heart) for implanting the prosthetic valve within the native tricuspid valve, the native pulmonary valve, or the pulmonary artery.

In all delivery approaches, the delivery apparatus can be advanced over a guidewire previously inserted into a patient's vasculature. Moreover, the disclosed delivery approaches are not intended to be limited. Any of the prosthetic valves disclosed herein can be implanted using any of various delivery procedures and delivery devices known in the art.

### Examples of the Disclosed Technology

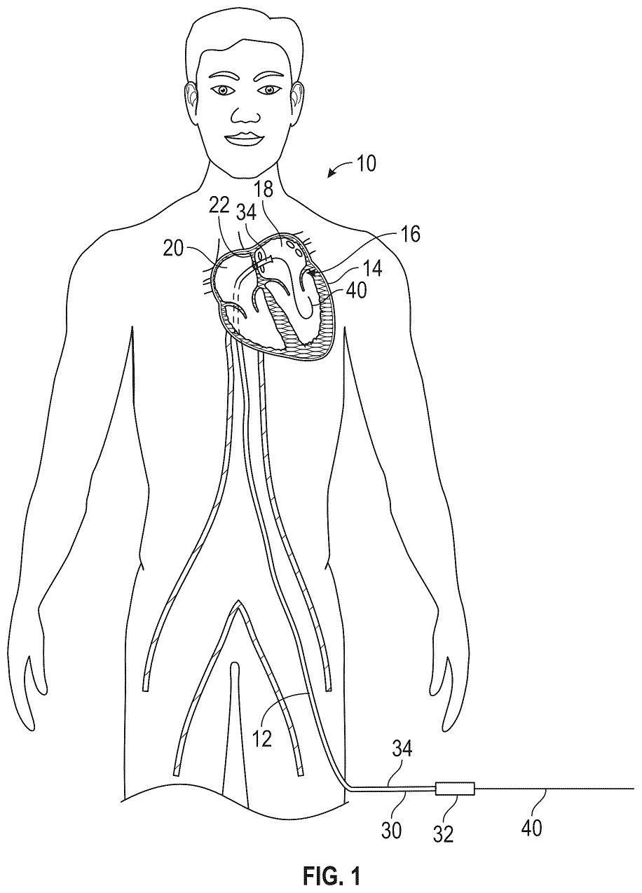

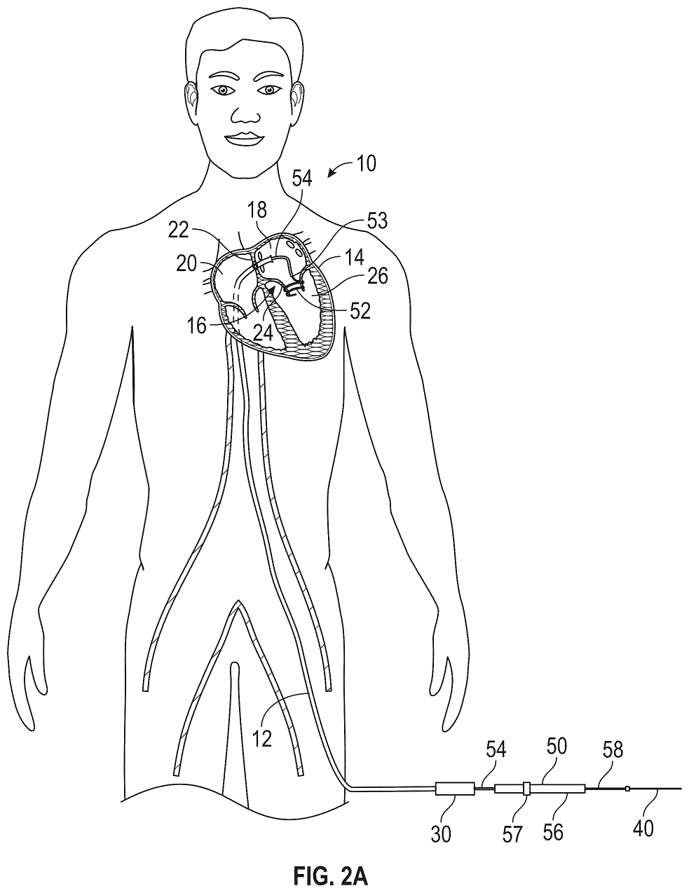

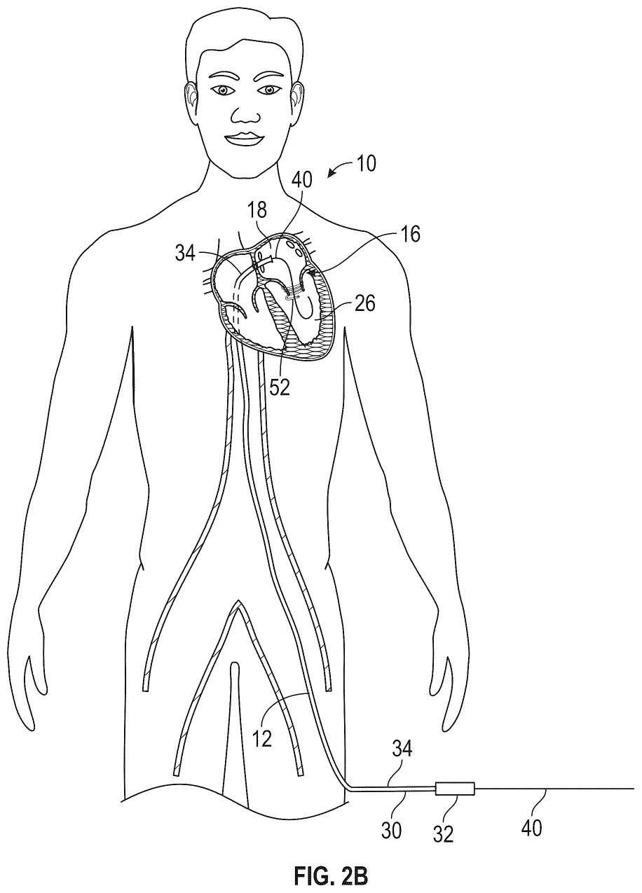

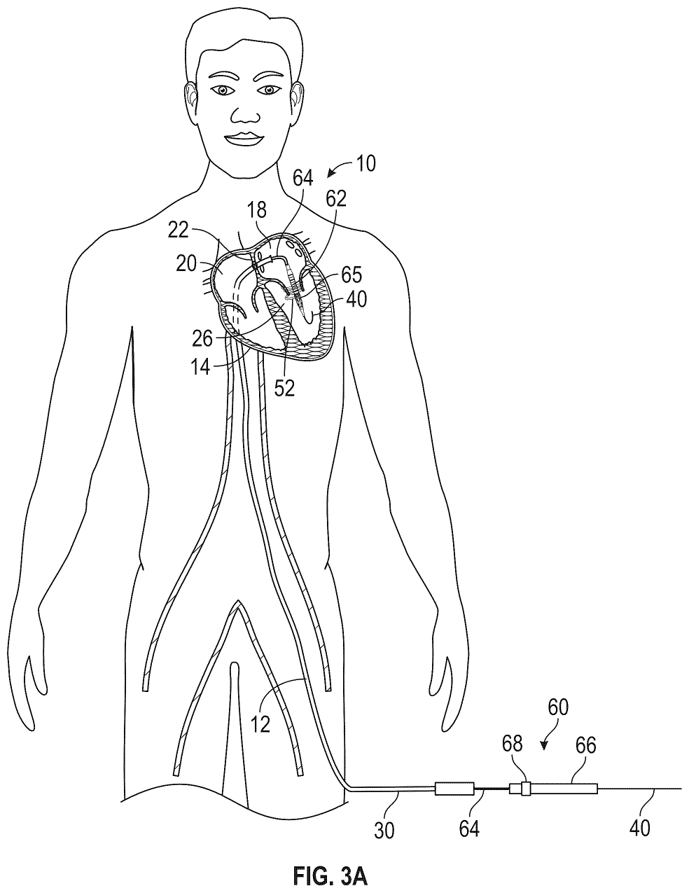

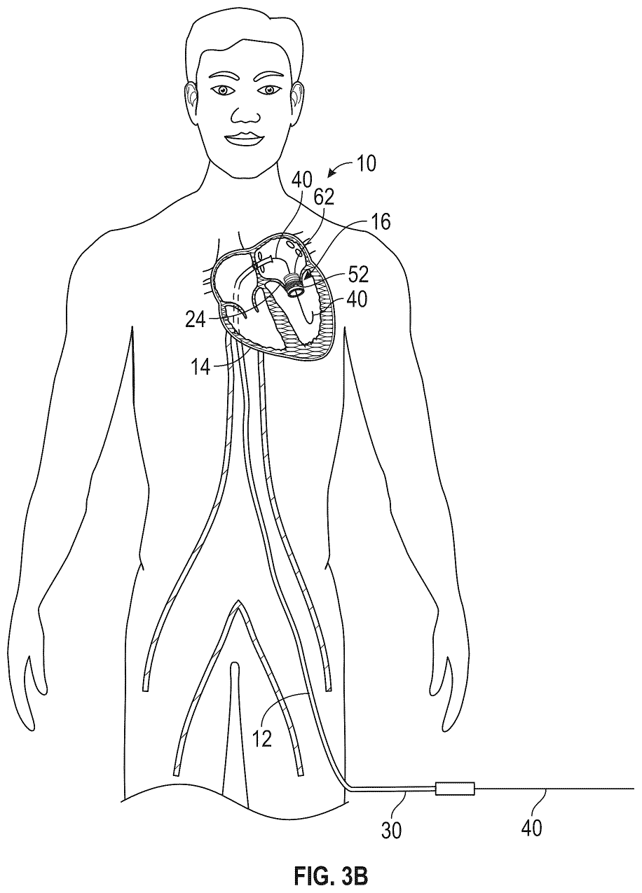

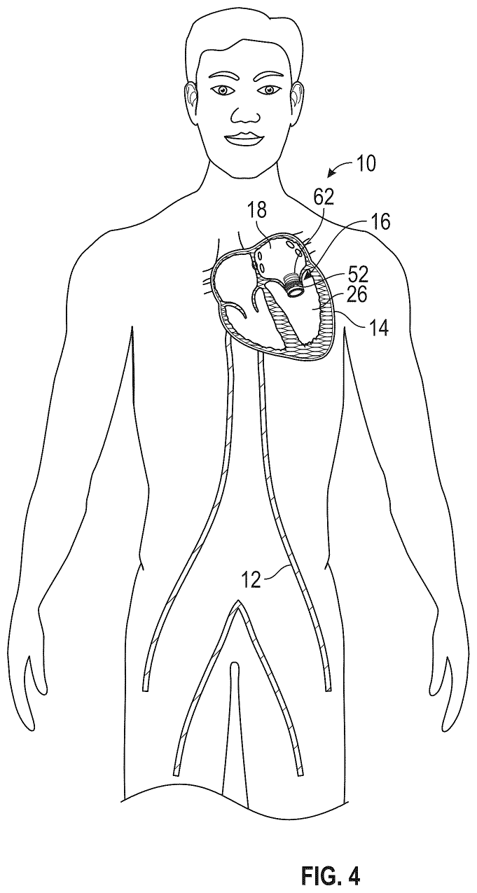

FIGS. 1-4 depict an exemplary transcatheter heart valve replacement procedure (e.g., a mitral valve replacement procedure) which utilizes a docking device 52 and a prosthetic heart valve 62, according to one example. During the procedure, a user first creates a pathway to a patient's native heart valve using a guide catheter 30 (FIG. 1). The user then delivers and implants the docking device 52 at the patient's native heart valve using a docking device delivery apparatus 50 (FIG. 2A) and then removes the docking device delivery apparatus 50 from the patient 10 after implanting the docking device 52 (FIG. 2B). The user then implants the prosthetic heart valve 62 within the implanted docking device 52 using a prosthetic valve delivery apparatus 60 (FIG. 3A). Thereafter, the user removes the prosthetic valve delivery apparatus 60 from the patient 10 (FIG. 3B), as well as the guide catheter 30 (FIG. 4).

FIG. 1 depicts a first stage in a mitral valve replacement procedure, according to one example, where the guide catheter 30 and a guidewire 40 are inserted into a blood vessel 12 of a patient 10 and navigated through the blood vessel 12, into a heart 14 of the patient 10, and toward the native mitral valve 16. Together, the guide catheter 30 and the guidewire 40 can provide a path for the docking device delivery apparatus 50 and the prosthetic valve delivery apparatus 60 to be navigated through and along, to the implantation site (the native mitral valve 16 or native mitral valve annulus).

Initially, the user may first make an incision in the patient's body to access the blood vessel 12. For example, in the example illustrated in FIG. 1, the user may make an incision in the patient's groin to access a femoral vein. Thus, in such examples, the blood vessel 12 may be a femoral vein.

After making the incision at the blood vessel 12, the user may insert the guide catheter 30, the guidewire 40, and/or additional devices (such as an introducer device or transseptal puncture device) through the incision and into the blood vessel 12. The guide catheter 30 (which can also be referred to as an “introducer device,” “introducer,” or “guide sheath”) is configured to facilitate the percutaneous introduction of various implant delivery devices (e.g., the docking device delivery apparatus 50 and the prosthetic valve delivery apparatus 60) into and through the blood vessel 12 and may extend through the blood vessel 12 and into the heart 14 but may stop short of the native mitral valve 16. The guide catheter 30 can comprise a handle 32 and a shaft 34 extending distally from the handle 32. The shaft 34 can extend through the blood vessel 12 and into the heart 14 while the handle 32 remains outside the body of the patient 10 and can be operated by the user in order to manipulate the shaft 34 (FIG. 1).

The guidewire 40 is configured to guide the delivery apparatuses (e.g., the guide catheter 30, the docking device delivery apparatus 50, the prosthetic valve delivery apparatus 60, additional catheters, or the like) and their associated devices (e.g., docking device, prosthetic heart valve, and the like) to the implantation site within the heart 14, and thus may extend all the way through the blood vessel 12 and into a left atrium 18 of the heart 14 (and in some examples, through the native mitral valve 16 and into a left ventricle of the heart 14) (FIG. 1).

In some instances, a transseptal puncture device or catheter can be used to initially access the left atrium 18, prior to inserting the guidewire 40 and the guide catheter 30. For example, after making the incision to the blood vessel 12, the user may insert a transseptal puncture device through the incision and into the blood vessel 12. The user may guide the transseptal puncture device through the blood vessel 12 and into the heart 14 (e.g., through the femoral vein and into the right atrium 20). The user can then make a small incision in an atrial septum 22 of the heart 14 to allow access to the left atrium 18 from the right atrium 20. The user can then insert and advance the guidewire 40 through the transseptal puncture device within the blood vessel 12 and through the incision in the atrial septum 22 into the left atrium 18. Once the guidewire 40 is positioned within the left atrium 18 and/or the left ventricle 26, the transseptal puncture device can be removed from the patient 10. The user can then insert the guide catheter 30 into the blood vessel 12 and advance the guide catheter 30 into the left atrium 18 over the guidewire 40 (FIG. 1).

In some instances, an introducer device can be inserted through a lumen of the guide catheter 30 prior to inserting the guide catheter 30 into the blood vessel 12. In some instances, the introducer device can include a tapered end that extends out a distal tip of the guide catheter 30 and that is configured to guide the guide catheter 30 into the left atrium 18 over the guidewire 40. Additionally, in some instances the introducer device can include a proximal end portion that extends out a proximal end of the guide catheter 30. Once the guide catheter 30 reaches the left atrium 18, the user can remove the introducer device from inside the guide catheter 30 and the patient 10. Thus, only the guide catheter 30 and the guidewire 40 remain inside the patient 10. The guide catheter 30 is then in position to receive an implant delivery apparatus and help guide it to the left atrium 18, as described further below.

FIG. 2A depicts a second stage in the exemplary mitral valve replacement procedure where a docking device 52 is being implanted at the native mitral valve 16 of the heart 14 of the patient 10 using a docking device delivery apparatus 50 (which may also be referred to as an “implant catheter” and/or a “docking device delivery device”).

In general, the docking device delivery apparatus 50 comprises a delivery shaft 54, a handle 56, and a pusher assembly 58. The delivery shaft 54 is configured to be advanced through the patient's vasculature (e.g., blood vessel 12) and to the implantation site (e.g., native mitral valve 16) by the user and may be configured to retain the docking device 52 in a distal end portion 53 of the delivery shaft 54. In some examples, the distal end portion 53 of the delivery shaft 54 retains the docking device 52 therein in a straightened delivery configuration.

The handle 56 of the docking device delivery apparatus 50 is configured to be gripped and/or otherwise held by the user, outside the body of the patient 10, to advance the delivery shaft 54 through the patient's vasculature (e.g., blood vessel 12).

In some examples, the handle 56 can comprise one or more articulation members 57 (or rotatable knobs) that are configured to aid in navigating the delivery shaft 54 through the blood vessel 12. For example, the one or more articulation members 57 can comprise one or more of knobs, buttons, wheels, and/or other types of physically adjustable control members that are configured to be adjusted by the user to flex, bend, twist, turn, and/or otherwise articulate a distal end portion 53 of the delivery shaft 54 to aid in navigating the delivery shaft 54 through the blood vessel 12 and within the heart 14.

The pusher assembly 58 can be configured to deploy and/or implant the docking device 52 at the implantation site (e.g., the native mitral valve 16). For example, the pusher assembly 58 is configured to be adjusted by the user to push the docking device 52 out of the distal end portion 53 of the delivery shaft 54. A shaft of the pusher assembly 58 can extend through the delivery shaft 54 and can be disposed adjacent to the docking device 52 within the delivery shaft 54. In some examples, and as described in more detail below, the docking device 52 can be releasably coupled to the shaft of the pusher assembly 58 via a connection mechanism of the docking device delivery apparatus 50 such that the docking device 52 can be released after being deployed at the native mitral valve 16.

Further details of the docking device delivery apparatus and its variants are described in International Publication No. WO2020/247907, which is incorporated by reference herein in its entirety.

Referring again to FIG. 2A, after the guide catheter 30 is positioned within the left atrium 18, the user may insert the docking device delivery apparatus 50 (e.g., the delivery shaft 54) into the patient 10 by advancing the delivery shaft 54 of the docking device delivery apparatus 50 through the guide catheter 30 and over the guidewire 40. In some examples, the guidewire 40 can be at least partially retracted away from the left atrium 18 and into the guide catheter 30. The user may then continue to advance the delivery shaft 54 of the docking device delivery apparatus 50 through the blood vessel 12 along the guidewire 40 until the delivery shaft 54 reaches the left atrium 18, as illustrated in FIG. 2A. Specifically, the user may advance the delivery shaft 54 of the docking device delivery apparatus 50 by gripping and exerting a force on (e.g., pushing) the handle 56 of the docking device delivery apparatus 50 toward the patient 10. While advancing the delivery shaft 54 through the blood vessel 12 and the heart 14, the user may adjust the one or more articulation members 57 of the handle 56 to navigate the various turns, corners, constrictions, and/or other obstacles in the blood vessel 12 and the heart 14.

Once the delivery shaft 54 reaches the left atrium 18 and extends out of a distal end of the guide catheter 30, the user can position the distal end portion 53 of the delivery shaft 54 at and/or near the posteromedial commissure of the native mitral valve 16 using the handle 56 (e.g., the articulation members 57). The user may then push the docking device 52 out of the distal end portion 53 of the delivery shaft 54 with the shaft of the pusher assembly 58 to deploy and/or implant the docking device 52 within the annulus of the native mitral valve 16.

In some examples, the docking device 52 may be constructed from, formed of, and/or comprise a shape memory material, and as such, may return to its original, pre-formed shape when it exits the delivery shaft 54 and is no longer constrained by the delivery shaft 54. As one example, the docking device 52 may originally be formed as a coil, and thus may wrap around leaflets 24 of the native mitral valve 16 as it exits the delivery shaft 54 and returns to its original coiled configuration.

After pushing a ventricular portion of the docking device 52 (e.g., the portion of the docking device 52 shown in FIG. 2A that is configured to be positioned within a left ventricle 26 and/or on the ventricular side of the native mitral valve 16), the user may then deploy the remaining portion of the docking device 52 (e.g., an atrial portion of the docking device 52) from the delivery shaft 54 within the left atrium 18 by retracting the delivery shaft 54 away from the posteromedial commissure of the native mitral valve 16.

After deploying and implanting the docking device 52 at the native mitral valve 16, the user may disconnect the docking device delivery apparatus 50 from the docking device 52. Once the docking device 52 is disconnected from the docking device delivery apparatus 50, the user may retract the docking device delivery apparatus 50 out of the blood vessel 12 and away from the patient 10 so that the user can deliver and implant a prosthetic heart valve 62 within the implanted docking device 52 at the native mitral valve 16.

FIG. 2B depicts this third stage in the mitral valve replacement procedure, where the docking device 52 has been fully deployed and implanted at the native mitral valve 16 and the docking device delivery apparatus 50 (including the delivery shaft 54) has been removed from the patient 10 such that only the guidewire 40 and the guide catheter 30 remain inside the patient 10. In some examples, after removing the docking device delivery apparatus, the guidewire 40 can be advanced out of the guide catheter 30, through the implanted docking device 52 at the native mitral valve 16, and into the left ventricle 26 (FIG. 2A). As such, the guidewire 40 can help to guide the prosthetic valve delivery apparatus 60 through the annulus of the native mitral valve 16 and at least partially into the left ventricle 26.

As illustrated in FIG. 2B, the docking device 52 can comprise a plurality of turns (or coils) that wrap around the leaflets 24 of the native mitral valve 16 (within the left ventricle 26). The implanted docking device 52 has a more cylindrical shape than the annulus of the native mitral valve 16, thereby providing a geometry that more closely matches the shape or profile of the prosthetic heart valve to be implanted. As a result, the docking device 52 can provide a tighter fit, and thus a better seal, between the prosthetic heart valve and the native mitral valve 16, as described further below.

FIG. 3A depicts a fourth stage in the mitral valve replacement procedure where the user is delivering and/or implanting a prosthetic heart valve 62 (which can also be referred to herein as a “transcatheter prosthetic heart valve” or “THV” for short, “replacement heart valve,” and/or “prosthetic mitral valve”) within the docking device 52 using a prosthetic valve delivery apparatus 60.

As shown in FIG. 3A, the prosthetic valve delivery apparatus 60 can comprise a delivery shaft 64 and a handle 66, the delivery shaft 64 extending distally from the handle 66. The delivery shaft 64 is configured to extend into the patient's vasculature to deliver, implant, expand, and/or otherwise deploy the prosthetic heart valve 62 within the docking device 52 at the native mitral valve 16. The handle 66 is configured to be gripped and/or otherwise held by the user to advance the delivery shaft 64 through the patient's vasculature.

In some examples, the handle 66 can comprise one or more articulation members 68 that are configured to aid in navigating the delivery shaft 64 through the blood vessel 12 and the heart 14. Specifically, the articulation member(s) 68 can comprise one or more of knobs, buttons, wheels, and/or other types of physically adjustable control members that are configured to be adjusted by the user to flex, bend, twist, turn, and/or otherwise articulate a distal end portion of the delivery shaft 64 to aid in navigating the delivery shaft 64 through the blood vessel 12 and into the left atrium 18 and left ventricle 26 of the heart 14.

In some examples, the prosthetic valve delivery apparatus 60 can include an expansion mechanism 65 that is configured to radially expand and deploy the prosthetic heart valve 62 at the implantation site. In some instances, as shown in FIG. 3A, the expansion mechanism 65 can comprise an inflatable balloon that is configured to be inflated to radially expand the prosthetic heart valve 62 within the docking device 52. The inflatable balloon can be coupled to the distal end portion of the delivery shaft 64.

In other examples, the prosthetic heart valve 62 can be self-expanding and can be configured to radially expand on its own upon removable of a sheath or capsule covering the radially compressed prosthetic heart valve 62 on the distal end portion of the delivery shaft 64. In still other examples, the prosthetic heart valve 62 can be mechanically expandable and the prosthetic valve delivery apparatus 60 can include one or more mechanical actuators (e.g., the expansion mechanism) configured to radially expand the prosthetic heart valve 62.

As shown in FIG. 3A, the prosthetic heart valve 62 is mounted around the expansion mechanism 65 (the inflatable balloon) on the distal end portion of the delivery shaft 64, in a radially compressed configuration.

To navigate the distal end portion of the delivery shaft 64 to the implantation site, the user can insert the prosthetic valve delivery apparatus 60 (the delivery shaft 64) into the patient 10 through the guide catheter 30 and over the guidewire 40. The user can continue to advance the prosthetic valve delivery apparatus 60 along the guidewire 40 (through the blood vessel 12) until the distal end portion of the delivery shaft 64 reaches the native mitral valve 16, as illustrated in FIG. 3A. More specifically, the user can advance the delivery shaft 64 of the prosthetic valve delivery apparatus 60 by gripping and exerting a force on (e.g., pushing) the handle 66. While advancing the delivery shaft 64 through the blood vessel 12 and the heart 14, the user can adjust the one or more articulation members 68 of the handle 66 to navigate the various turns, corners, constrictions, and/or other obstacles in the blood vessel 12 and heart 14.

The user can advance the delivery shaft 64 along the guidewire 40 until the radially compressed prosthetic heart valve 62 mounted around the distal end portion of the delivery shaft 64 is positioned within the docking device 52 and the native mitral valve 16. In some examples, as shown in FIG. 3A, a distal end of the delivery shaft 64 and a least a portion of the radially compressed prosthetic heart valve 62 can be positioned within the left ventricle 26.

Once the radially compressed prosthetic heart valve 62 is appropriately positioned within the docking device 52 (FIG. 3A), the user can manipulate one or more actuation mechanisms of the handle 66 of the prosthetic valve delivery apparatus 60 to actuate the expansion mechanism 65 (e.g., inflate the inflatable balloon), thereby radially expanding the prosthetic heart valve 62 within the docking device 52.

FIG. 3B shows a fifth stage in the mitral valve replacement procedure where the prosthetic heart valve 62 in its radially expanded configuration and implanted within the docking device 52 in the native mitral valve 16. As shown in FIG. 3B, the prosthetic heart valve 62 is received and retained within the docking device 52. Thus, the docking device 52 aids in anchoring the prosthetic heart valve 62 within the native mitral valve 16. The docking device 52 can enable better sealing between the prosthetic heart valve 62 and the leaflets 24 of the native mitral valve 16 to reduce paravalvular leakage around the prosthetic heart valve 62.

As also shown in FIG. 3B, after the prosthetic heart valve 62 has been fully deployed and implanted within the docking device 52 at the native mitral valve 16, the prosthetic valve delivery apparatus 60 (including the delivery shaft 64) is removed from the patient 10 such that only the guidewire 40 and the guide catheter 30 remain inside the patient 10.

FIG. 4 depicts a sixth stage in the mitral valve replacement procedure, where the guidewire 40 and the guide catheter 30 have been removed from the patient 10.

Although FIGS. 1-4 specifically depict a mitral valve replacement procedure, it should be appreciated that the same and/or similar procedure may be utilized to replace other heart valves (e.g., tricuspid, pulmonary, and/or aortic valves). Further, the same and/or similar delivery apparatuses (e.g., docking device delivery apparatus 50, prosthetic valve delivery apparatus 60, guide catheter 30, and/or guidewire 40), docking devices (e.g., docking device 52), replacement heart valves (e.g., prosthetic heart valve 62), and/or components thereof may be utilized for replacing these other heart valves.

For example, when replacing a native tricuspid valve, the user may also access the right atrium 20 via a femoral vein but may not need to cross the atrial septum 22 into the left atrium 18. Instead, the user may leave the guidewire 40 in the right atrium 20 and perform the same and/or similar docking device implantation process at the tricuspid valve. Specifically, the user may push the docking device 52 out of the delivery shaft 54 around the ventricular side of the tricuspid valve leaflets, release the remaining portion of the docking device 52 from the delivery shaft 54 within the right atrium 20, and then remove the delivery shaft 54 of the docking device delivery apparatus 50 from the patient 10. The user may then advance the guidewire 40 through the tricuspid valve into the right ventricle and perform the same and/or similar prosthetic heart valve implantation process at the tricuspid valve, within the docking device 52. Specifically, the user may advance the delivery shaft 64 of the prosthetic valve delivery apparatus 60 through the patient's vasculature along the guidewire 40 until the prosthetic heart valve 62 is positioned/disposed within the docking device 52 and the tricuspid valve. The user may then expand the prosthetic heart valve 62 within the docking device 52 before removing the prosthetic valve delivery apparatus 60 from the patient 10. In another example, the user may perform the same and/or similar process to replace the aortic valve but may access the aortic valve from the outflow side of the aortic valve via a femoral artery.

Further, although FIGS. 1-4 depict a mitral valve replacement procedure that accesses the native mitral valve 16 from the left atrium 18 via the right atrium 20 and femoral vein, it should be appreciated that the native mitral valve 16 may alternatively be accessed from the left ventricle 26. For example, the user may access the native mitral valve 16 from the left ventricle 26 via the aortic valve by advancing one or more delivery apparatuses through an artery to the aortic valve, and then through the aortic valve into the left ventricle 26.

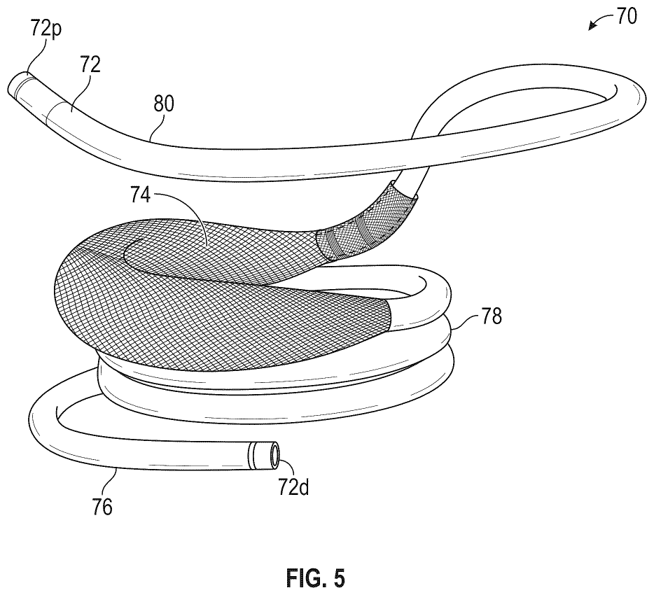

FIG. 5 illustrates a docking device 70, according to one example. The docking device 70 can, for example, be used as the docking device 52 in a prosthetic valve implantation procedure, as described above with reference to FIGS. 1-4. As depicted in FIG. 5, the docking device in its deployed configuration can be configured to receive and secure a prosthetic valve within the docking device, thereby securing the prosthetic valve at the native valve annulus.

The docking device 70 can comprise a coil 72 and a guard member 74 covering at least a portion of the coil 72. In certain examples, the coil 72 can include a shape memory material (e.g., nickel titanium alloy or “Nitinol”) such that the docking device 70 (and the coil 72) can move from a substantially straight configuration (or delivery configuration) when disposed within the delivery shaft 54 of the delivery apparatus 50 to a helical, deployed configuration after being removed from the delivery shaft 54.

The coil 72 has a proximal end 72p and a distal end 72d (which also respectively define the proximal and distal ends of the docking device 70). When being disposed within the delivery shaft 54 (e.g., during delivery of the docking device 70 into the vasculature of a patient), a body of the coil 72 between the proximal end 72p and distal end 72d can form a generally straight delivery configuration (i.e., without any coiled or looped portions, but can be flexed or bent) so as to maintain a small radial profile when moving through a patient's vasculature. After being removed from the delivery shaft 54 and deployed at an implant position, the coil 72 can move from the delivery configuration to the helical deployed configuration and wrap around native tissue adjacent the implant position. For example, when implanting the docking device at the location of a native valve, the coil 72 can be configured to surround native leaflets of the native valve (and the chordae tendineae that connects native leaflets to adjacent papillary muscles, if present).

The docking device 70 can be releasably coupled to the docking device delivery apparatus 50. For example, in certain examples, the docking device 70 can be coupled to a delivery apparatus (as described above) via a release suture that can be configured to be tied to the docking device 70 and cut for removal.

As shown in FIG. 5, the coil 72 in the deployed configuration can include a leading turn 76 (or “leading coil”), a central region 78, and a stabilization turn 80 (or “stabilization coil”) around a central longitudinal axis. The central region 78 can possess one or more helical turns having substantially equal inner diameters. The leading turn 76 can extend from a distal end of the central region 78 and has a diameter greater than the diameter of the central region 78, in the illustrated example. The stabilization turn 80 can extend from a proximal end of the central region 78 and has a diameter greater than the diameter of the central region 78, in the illustrated example.

Further details of the docking device and its variants are described in International Application No. PCT/US2021/056150, which is incorporated by reference herein in its entirety.

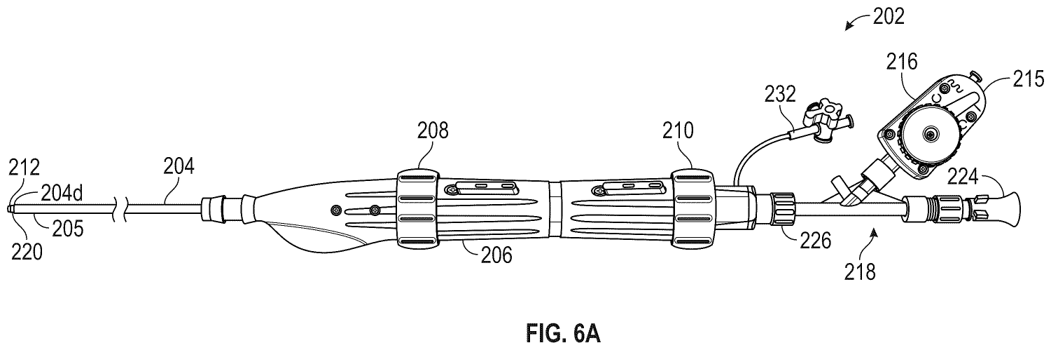

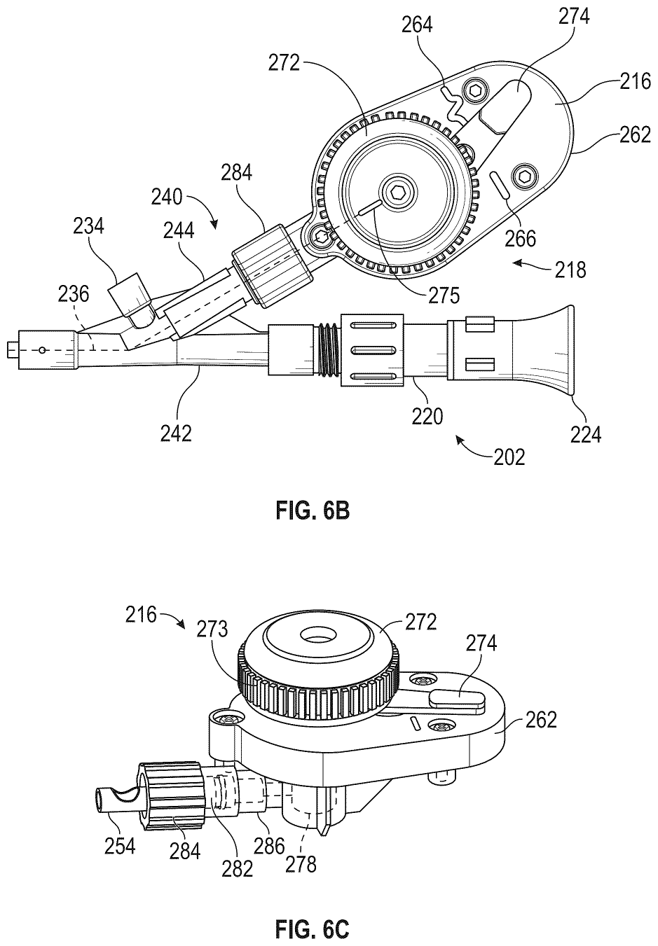

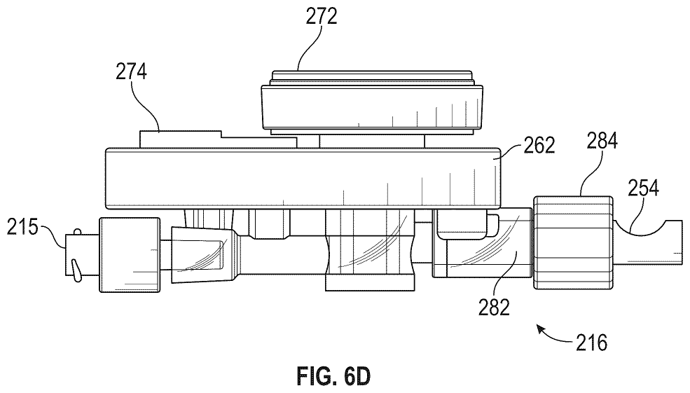

FIG. 6A illustrates a delivery apparatus 200 configured to implant a docking device, such as docking device 70 (FIG. 5) described below or other docking devices, to a target implantation site in a patient, according to one example. For example, the delivery apparatus 200 can be used as the docking device delivery apparatus 50 in a prosthetic valve implantation procedure, as described above with reference to FIG. 2A. The delivery apparatus 200 can also be referred to as a “docking device delivery apparatus,” “dock delivery catheter,” or “dock delivery system.”

As shown, the delivery apparatus 200 can include a handle assembly 202 and a delivery shaft 204 (also referred to as the “delivery sheath” or “outer shaft” or “outer sheath”) extending distally from the handle assembly 202. The handle assembly 202 can include a handle 206 including one or more knobs, buttons, wheels, and/or other means for controlling and/or actuating one or more components of the delivery apparatus 200. For example, in some examples, as shown in FIG. 6A, the handle 206 can include knobs 208 and 210 which can be configured to steer or control flexing of the delivery apparatus 200 such as the delivery shaft 204 and/or the sleeve shaft 220 described below.

In certain examples, the delivery apparatus 200 can also include a 224 212 and a sleeve shaft 220, both of which can extend through an inner lumen of the delivery shaft 204 and have respective proximal end portions extending into the handle assembly 202.

As described below, a distal end portion (also referred to as “distal section”) of the sleeve shaft 220 can be configured to cover (e.g., surround) the docking device 70 (see FIG. 5). For example, the docking device 70 can be retained inside the sleeve shaft 220, which is further retained by a distal end portion 205 of the delivery shaft 204, when navigating through a patient's vasculature.

Additionally, the distal end portion 205 of the delivery shaft 204 can be configured to be steerable. In one example, by rotating a knob (e.g., 208 or 210) on the handle 206, a curvature of the distal end portion 205 can be adjusted so that the distal end portion 205 of the delivery shaft 204 can be oriented in a desired angle. For example, to implant the docking device 70 at the native mitral valve location, the distal end portion 205 of the delivery shaft 204 can be steered in the left atrium so that at least a portion of the sleeve shaft 220 and the docking device 70 retained therein can extend through the native mitral valve annulus at a location adjacent the posteromedial commissure.

In certain examples, the pusher shaft 212 and the sleeve shaft 220 can be coaxial with one another, at least within the delivery shaft 204. In addition, the delivery shaft 204 can be configured to be axially movable relative to the sleeve shaft 220 and the pusher shaft 212. As described further below, a distal end of the pusher shaft 212 can be inserted into a lumen of the sleeve shaft 220 and press against the proximal end of the docking device 70 retained inside the sleeve shaft 220.

After reaching a target implantation site, the docking device 70 can be deployed from the delivery shaft 204 by manipulating the pusher shaft 212 and sleeve shaft 220 using a hub assembly 218, as described further below. For example, by pushing the pusher shaft 212 in the distal direction while holding the delivery shaft 204 in place or retracting the delivery shaft 204 in the proximal direction while holding the pusher shaft 212 in place, or by pushing the pusher shaft 212 in the distal direction while simultaneously retracting the delivery shaft 204 in the proximal direction, the docking device 70 can be pushed out of a distal end 204d of the delivery shaft 204, thus permitting the docking device 70 to transition from a delivery configuration to a deployed configuration (see FIG. 5). In certain examples, the pusher shaft 212 and the sleeve shaft 220 can be actuated independently of each other.

During delivery, the docking device 70 can be coupled to the delivery apparatus 200 via a release suture (not shown in FIG. 6A), or other retrieval line comprising a string, yarn, or other material that can be configured to be tied around the docking device 70 and cut for removal, that extends through the pusher shaft 212. In one specific example, the release suture can extend through the delivery apparatus 200, e.g., through an inner lumen of the pusher shaft 212, to a suture lock assembly 216 of the delivery apparatus 200.

The handle assembly 202 can further include a hub assembly 218 to which the suture lock assembly 216 and a sleeve handle 224 are attached. The hub assembly 218 can be configured to independently control the pusher shaft 212 and the sleeve shaft 220 while the sleeve handle 224 can control an axial position of the sleeve shaft 220 relative to the pusher shaft 212. In this way, operation of the various components of the handle assembly 202 can actuate and control operation of the components arranged within the delivery shaft 204. In some examples, the hub assembly 218 can be coupled to the handle 206 via a connector 226.

The handle assembly 202 can further include one or more flush ports (e.g., flush port 232 is shown in FIG. 6A) to supply flush fluid to one or more lumens arranged within the delivery apparatus 200 (e.g., annular lumens arranged between coaxial components of the delivery apparatus 200).

FIGS. 6B-6D illustrate aspects of the handle assembly 202 and/or the suture lock assembly 216 of FIG. 6A in more detail. As shown, the hub assembly 218 can include a Y-shaped connector 240 (also referred to as an “adaptor”) having a straight section 242 (e.g., straight conduit) and at least one branch 244 (e.g., branch conduit), although, in some examples, it can include more than one branch. In some examples, the suture lock assembly 216 can be attached to the branch 244 and the sleeve handle (e.g., sleeve actuating handle) 224 can be arranged at a proximal end of the straight section 242.

As shown in FIGS. 6A-6D and introduced above, the delivery apparatus 200 can include a suture lock assembly 216 located on the branch 244 of the hub assembly 218 of the handle assembly 202. As described below, the example suture lock assembly 216 includes a release knob 284 that can be screwed onto an end of the Y-shaped connector 240.

As depicted in FIG. 6B, the hub assembly 218 can include a flushing port 234 configured to allow flushing of one or more lumens within the delivery apparatus 200 to sterilize and/or maintain hemostasis within the delivery apparatus 200.

As discussed above, a medical professional can deploy the docking device (e.g., the docking device 70) by manipulating the position of the handle assembly 202 and only adds one additional step to retract the sleeve by pulling back on the sleeve handle 224. The sleeve shaft (e.g., the sleeve shaft 220) and the pusher shaft (e.g., the pusher shaft 212) can be configured to work together such that they can be moved simultaneously together when deploying and positioning the docking device at the native valve (e.g., by moving the entire hub assembly 218 and/or the Y-shaped connector 240 forward and/or backward). Additionally, the sleeve shaft and the pusher shaft can also be configured to move independently so that the pusher shaft can hold the docking device in position while the sleeve shaft is retracted off from the docking device (e.g., by holding the hub assembly 218 and/or the connector 240 in place relative to the delivery shaft 204 and/or other parts of the delivery apparatus 200 and/or the docking device while pulling proximally on the sleeve handle 224 to withdraw the sleeve shaft). The sleeve shaft and the pusher shaft can be coaxial along some, all, or a majority of the delivery apparatus 200 to facilitate this working together, as explained above.

As shown in FIGS. 6A-6D, the suture lock assembly 216 can include a rotator 272 (which also may be referred to as a “rotatable handle”) to increase and decrease tension on a release suture 236 (shown in FIG. 6B) which can extend from the suture lock assembly 216, through the branch 244, and through the handle 222 and the delivery shaft 204 to connect to the docking device, as described above.

In certain examples, the release suture 236 can be wrapped around a spool 278 of the suture lock assembly 216 (see, e.g., FIG. 6C). The rotator 272 can be coupled to the spool 278, such that rotating the rotator 272 in a given direction can adjust (e.g., increase or decrease) tension on the release suture 236 traversing the delivery apparatus 200. Providing tension or slack to the release suture 236 via rotating the rotator 272 (and thus the spool 278) can bring the docking device 70 closer to or further away from the delivery apparatus 200, respectively. As used herein, the spool 278 also may be referred to as a spindle 278.

In some examples, the rotator 272 can include one or more gripping portions or grips that increase an ease of gripping the rotator 272 (e.g., via a user's hand), without slipping. For example, and as shown in FIG. 3, the rotator 272 can include a gripping portion 273 arranged around a circumference of the rotator 272 and that is configured to be gripped by a user during turning of the rotator 272. In some examples, the gripping portion 273 can include a plurality of ridges to increase traction and ease of gripping. In some examples, the gripping portion 273 can comprise a material having a lower durometer (e.g., reduced hardness) than a material that forms another portion of the rotator 272.

In some examples, as shown in FIG. 6B, the rotator 272 can include an indicator 275 to track a number of turns applied (or portions thereof), which in turn may be correlated to a degree of slack or tension in the release suture 236.

In some examples, the suture lock assembly 216 can further include a directional control mechanism which may include a directional selector 274 (e.g., in a form of a switch, as shown in FIGS. 6B-6C) that allows a medical practitioner or other user to select whether to increase or decrease slack in the release suture 236 traversing the delivery apparatus 200. For example, the directional selector 274 can be configured to allow a medical practitioner or other user to select a direction (e.g., increase or decrease tension), which will allow the rotator 272 to rotate in only one direction to prevent rotation in an incorrect direction.

In some examples, as shown in FIG. 6C, a housing 262 of the suture lock assembly 216 can include a first icon 264 indicating a slack position of the directional selector 274 and a second icon 266 indicating a tension position of the directional selector 274.

Additional details on the directional control mechanism for a suture lock assembly (such as the suture lock assembly 216) are described in International Patent Application No. PCT/US2020/36577, the disclosure of which is incorporated by reference herein.

In certain examples, the suture lock assembly 216 can include a connector or connecting portion to attach the suture lock assembly 216 to a handle assembly (e.g., the handle assembly 202). For example, the suture lock assembly 216 can include a release bar 282 that extends into and couples with the housing 262 of the suture lock assembly 216 (see, e.g., FIG. 6C). In some examples, the release bar 282 can be bonded to the housing 262 (e.g., via an adhesive, weld, or other non-removable fixing means). As shown in FIG. 6C, a release knob 284 can be arranged around a portion of the release bar 282, adjacent to a connecting portion 286 of the bottom housing 268. The release knob 284 can be configured to connect the suture lock assembly 216 to the adaptor 240 of the delivery apparatus.

As described above and depicted in FIG. 6B, the adaptor 240 can include a branch 244 and a straight section 242. In the depicted example, the release knob 284 can screw onto an end of the adaptor 240 (e.g., a proximal end of the branch 244) to secure the suture lock assembly 216 to the adaptor 240. In some examples, the shape, size, and/or configuration of the adaptor 240 may be different than shown in FIG. 6B and may change based on the delivery apparatus to which the suture lock assembly 216 is configured to be attached to (and used with).

In certain examples, when the release knob 284 is coupled to each of the adaptor 240 (or another adaptor of a delivery apparatus) and the release bar 282, the suture lock assembly 216 can be coupled to the delivery apparatus and a suture cutting section 254 can be covered by the adaptor 240 (see, e.g., FIG. 6C). In some examples, once the docking device (or other implant) is positioned in a desired position for release from the delivery apparatus, the release knob 284 can be unscrewed from the adaptor 240 to release the suture lock assembly 216 from the adaptor 240, and the suture lock assembly 216 can be pulled proximally away from the adaptor 240 to expose the suture cutting section 254. In alternate examples, rotation of the release knob 284 (e.g., to move the release knob 284 toward the housing 262) can expose the suture cutting section 254 without pulling the entire suture lock assembly 216 away from the adaptor 240.

The suture cutting section 254 can be configured to allow for a user or medical practitioner to cut the release suture 236 that traverses the length of a delivery apparatus, to allow for the disconnection of a docking device from the delivery apparatus upon its deployment at a target implantation site.

In some examples, once the release suture 236 is wrapped around the docking device or implant and routed through the delivery apparatus, through the release bar 282 (including across the suture cutting section 254), and into the housing 262, the two suture ends of the release suture 236 can be threaded through two apertures arranged in a bottom end of the spool 278 and then tied to complete a suture loop.

As depicted in FIGS. 6A and 6D, the suture lock assembly 216 can include a flushing port 215 to allow flushing of one or more lumens within the delivery apparatus to reduce thrombus formation between components of the delivery apparatus, maintain hemostasis within a delivery apparatus, and/or to sterilize a delivery apparatus. In certain cases, the flushing port 215 can be configured to allow flushing lumens independently if a single flush line becomes clogged and/or is not maintaining hemostasis in a delivery apparatus. In certain cases, the flushing port 215 can be an open port to allow constant flow through a delivery apparatus. In certain examples, the flushing port 215 can be configured as self-sealing such that fluids can be introduced into a delivery apparatus as needed by a practitioner without requiring constant flow. The flushing port 215 depicted in FIG. 6D allows for an additional flush line to be connected akin to multiple flushing ports, such as the flushing port 232 illustrated in FIG. 6A and/or the flushing port 234 illustrated in FIG. 6B, as discussed above. For simplicity, the flushing port 215 is not illustrated in FIGS. 6B-6C; however, it is to be understood that the suture lock assembly 216 illustrated in FIGS. 6B-6C also can include the flushing port 215.

Further details on delivery apparatus/catheters/systems (including various examples of the handle assembly) that are configured to deliver a docking device to a target implantation site can be found in International Application No. PCT/US2020/036577 and in U.S. Patent Publication Nos. 2018/0318079 and 2018/0263764, each of which is incorporated by reference herein in its entirety.

As discussed above, once the docking device 70 is implanted at the target implantation site, the release suture 236 is uncoupled from the docking device 70. In the example of FIGS. 6A-6D, this is accomplished by removing the suture lock assembly 216 from the hub assembly 218 to expose the suture cutting section 254 within which the release suture 236 may be cut. In other examples, and as discussed below, the suture lock assembly may be configured to remain coupled to the hub assembly 218 and/or the delivery apparatus 200 while the release suture is removed from the docking device. As described in more detail below, such devices may facilitate removal of the release suture 236 from the docking device 70.

FIG. 7 schematically illustrates an example of a suture lock assembly 300 that comprises a quick release suture mechanism 302 configured to facilitate releasing the release suture 236 from an implantable device (such as the docking device 70). The suture lock assembly 300 may include any features, characteristics, attributes, etc. disclosed herein with reference to the suture lock assembly 216, and vice versa. In particular, FIG. 7 illustrates an example in which the suture lock assembly 300 comprises a spool 340 (which may be similar to, or the same as, the spool 278) for collecting a length and/or adjusting a tension of the release suture 236. The suture lock assembly 300 additionally comprises a tensioner housing 350 that at least partially receives the spool 340.

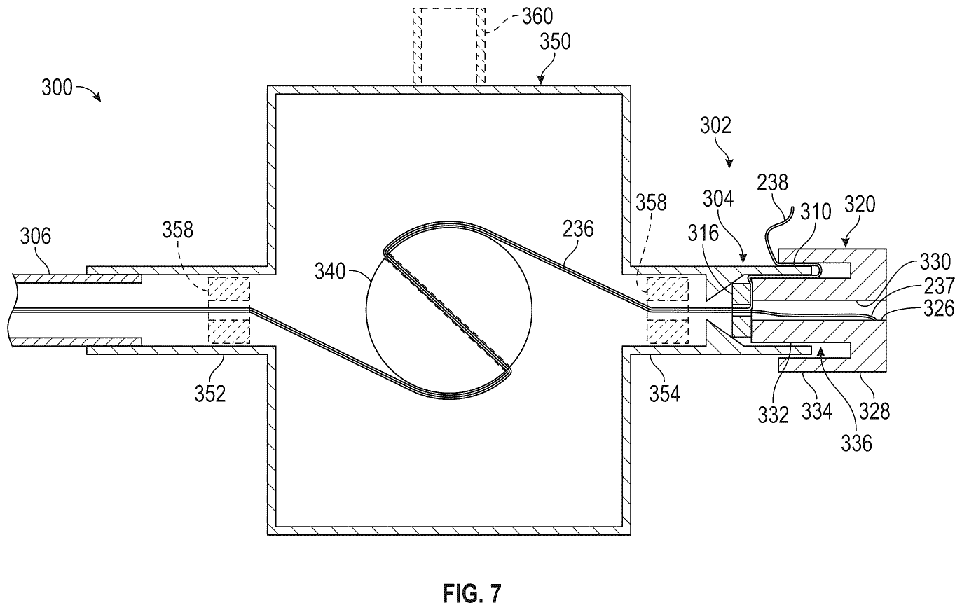

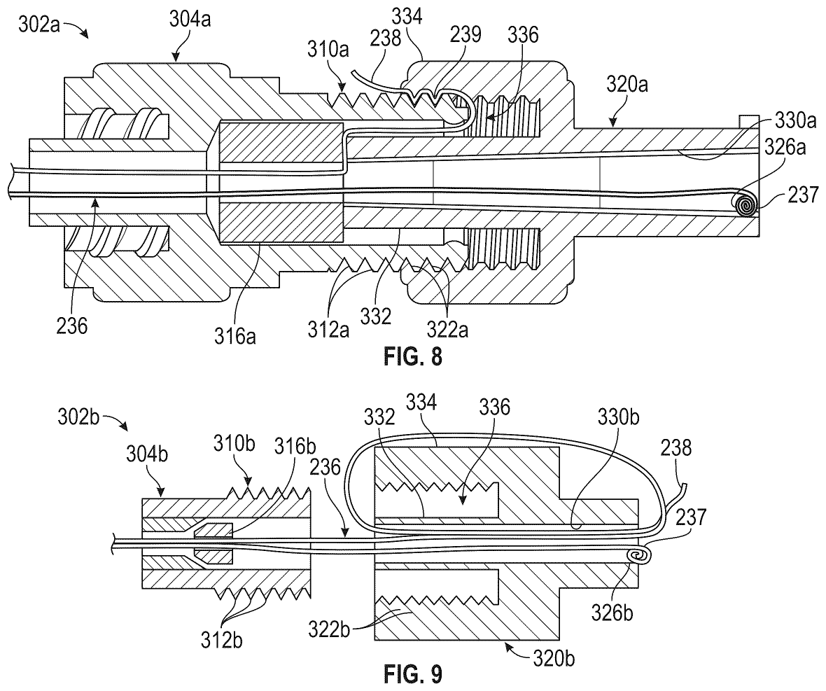

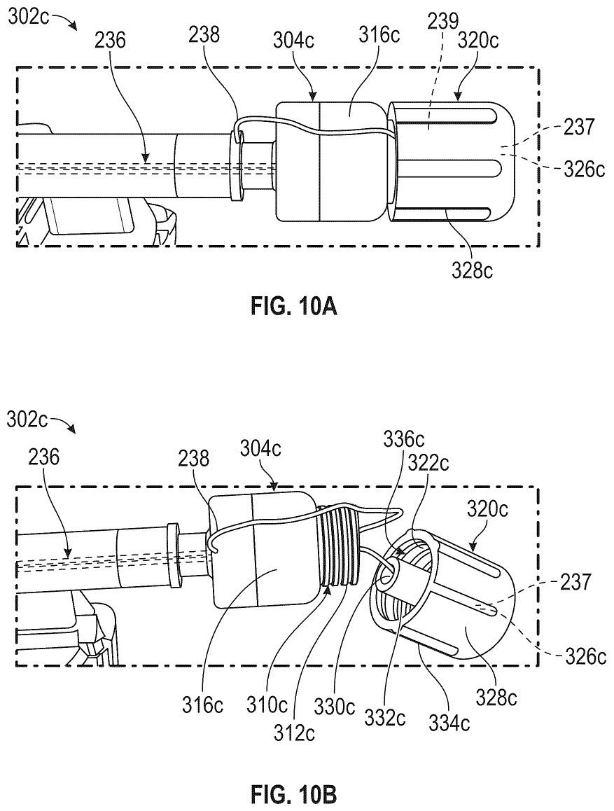

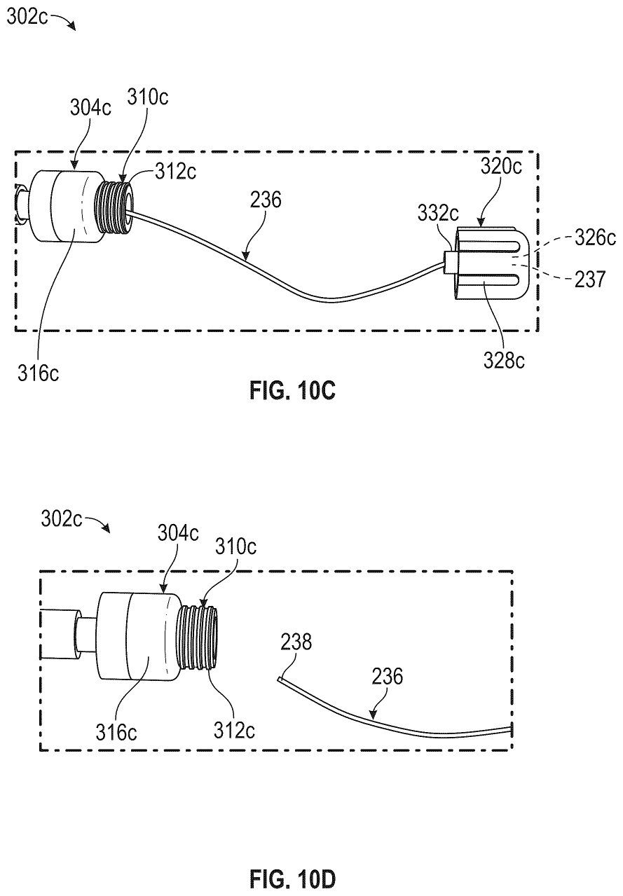

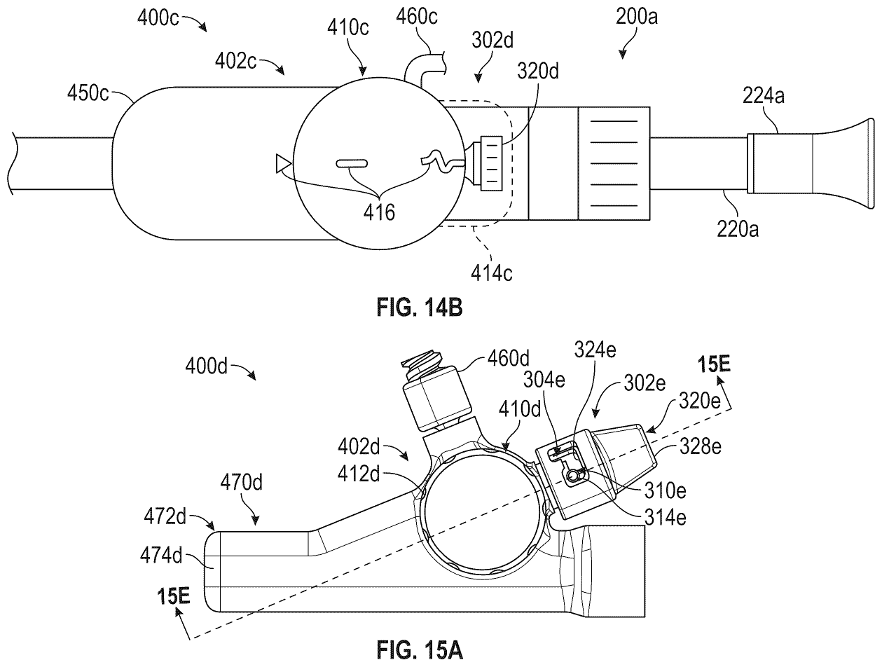

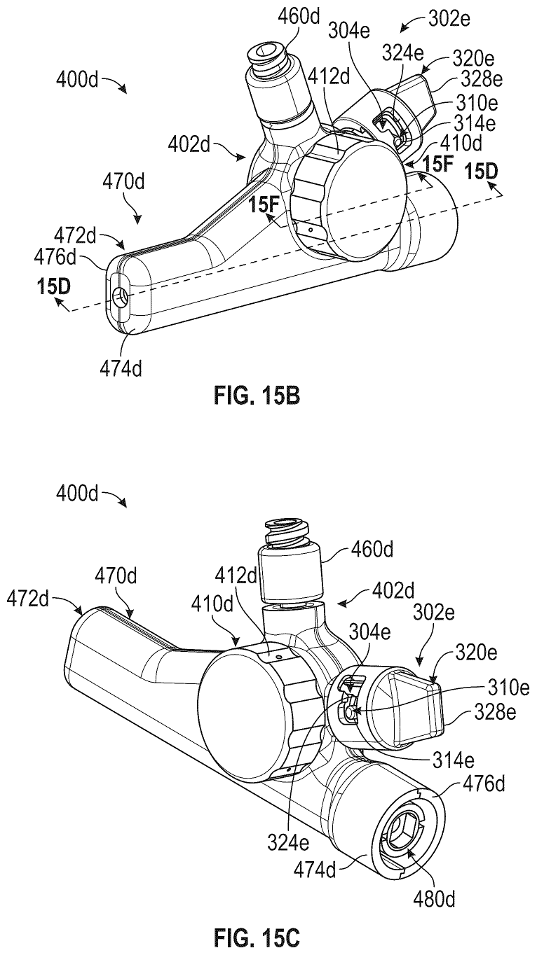

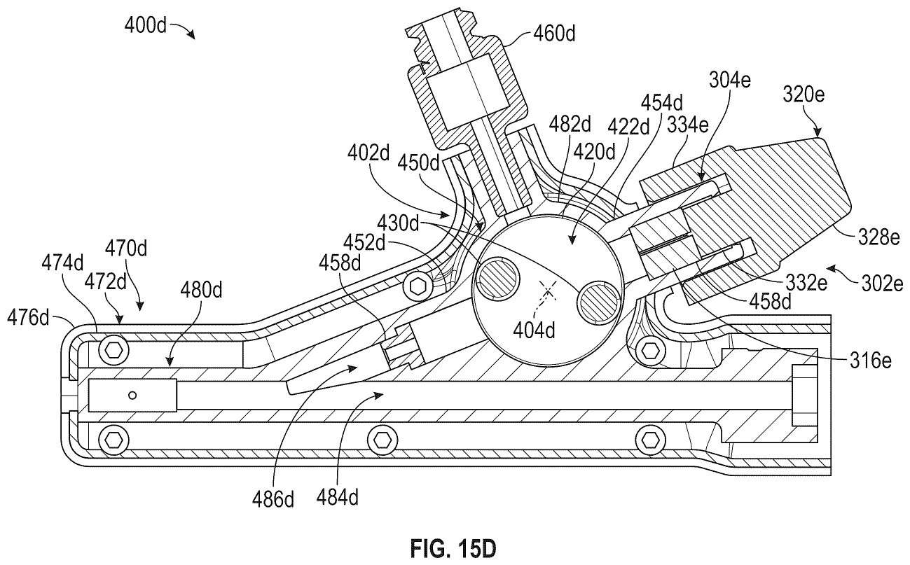

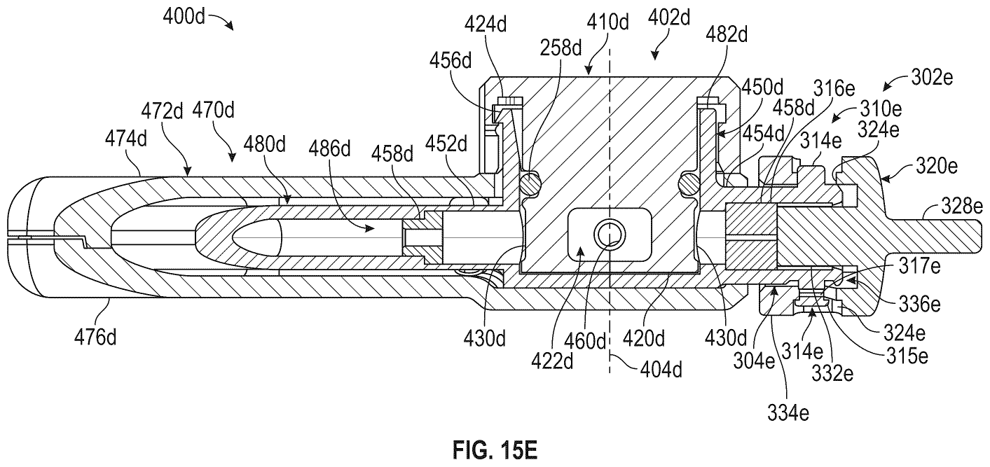

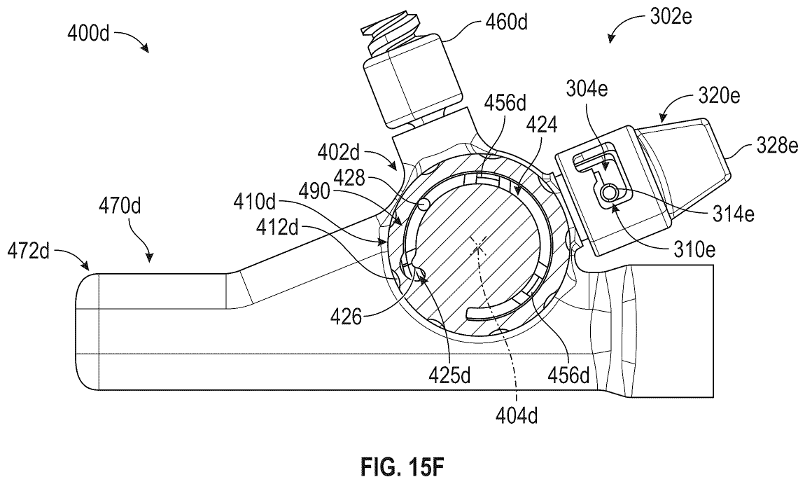

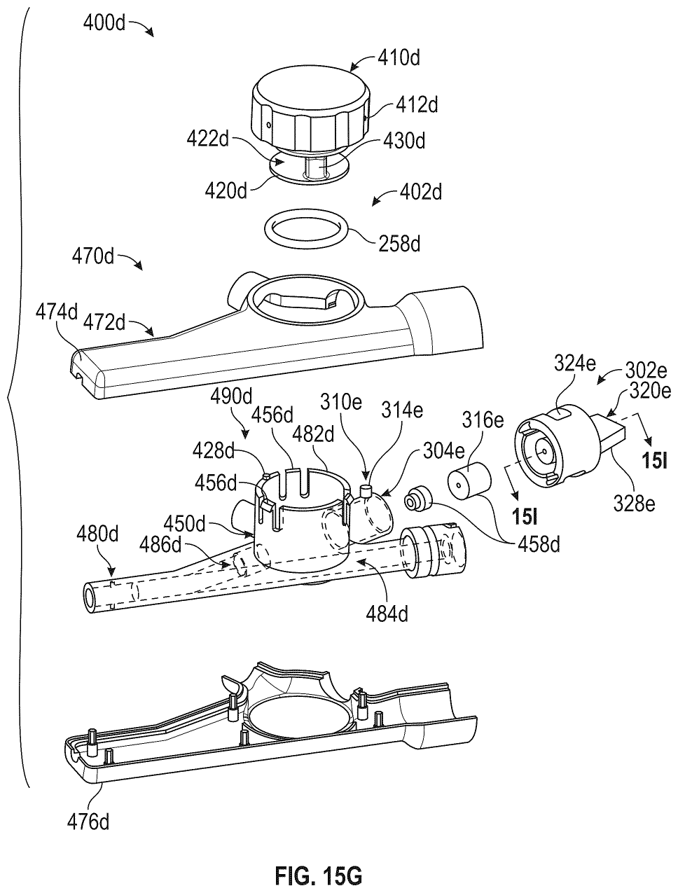

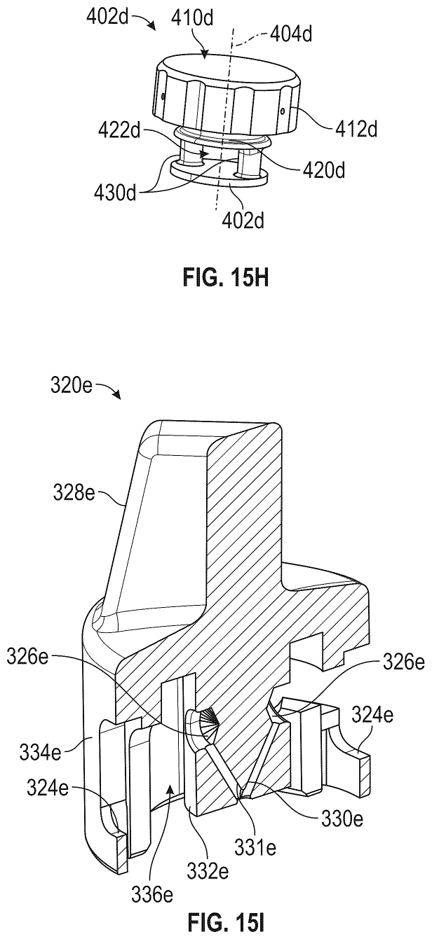

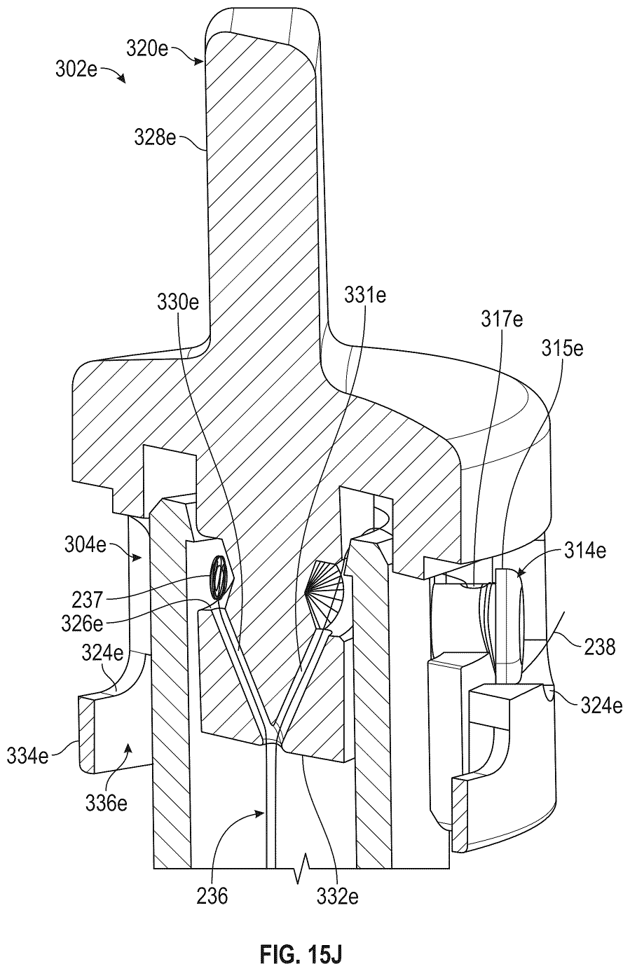

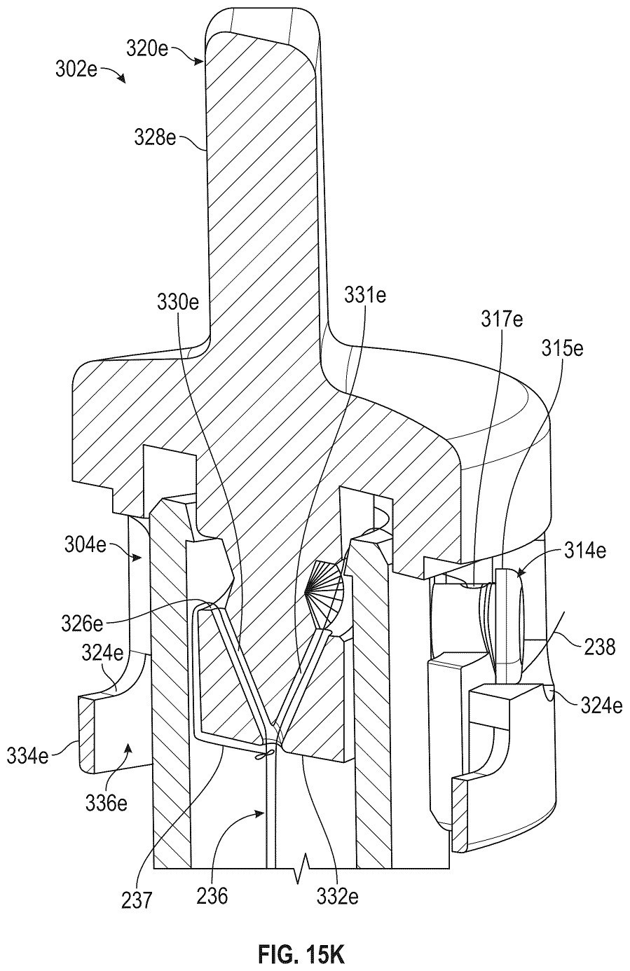

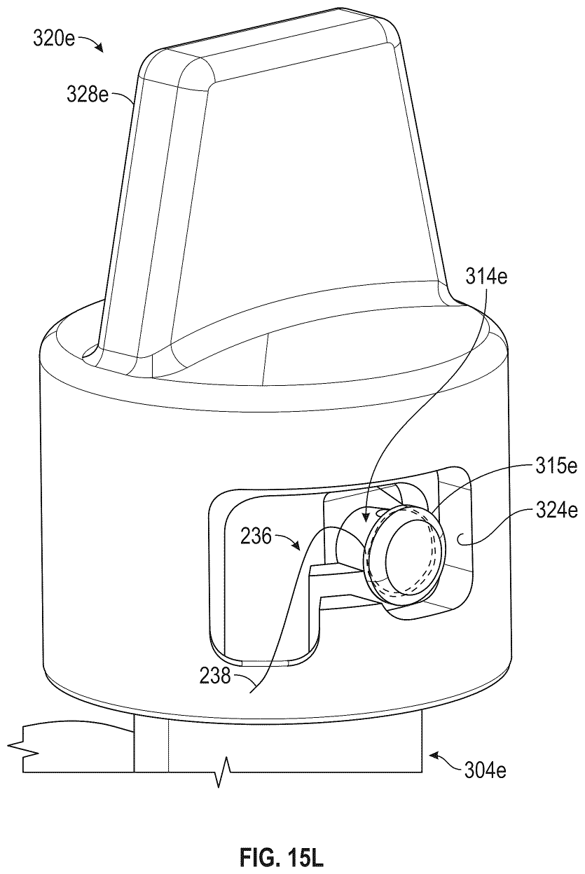

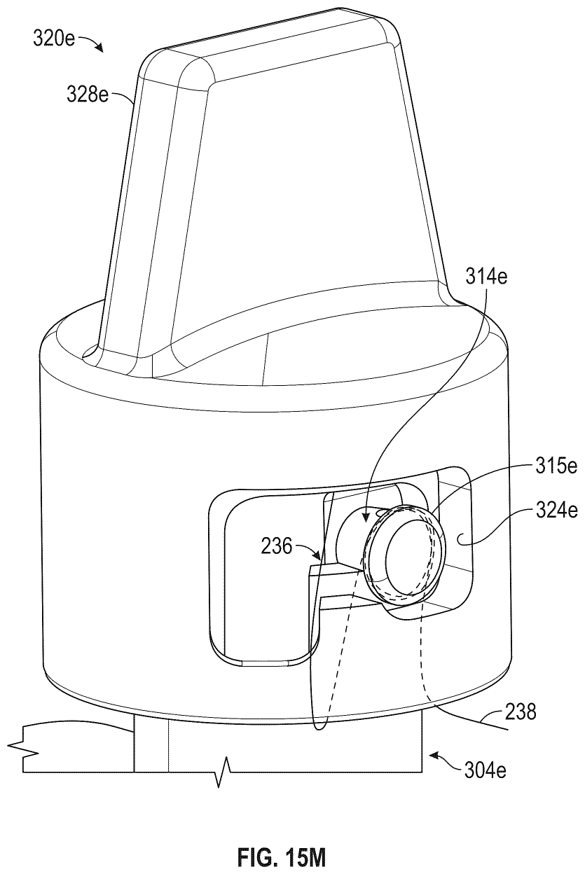

FIGS. 8-10, 14A-15G, and 15I-15M illustrate several additional examples of quick release suture mechanisms 302, as described in more detail below. In particular, FIG. 8 illustrates a first example quick release suture mechanism 302a, while FIG. 9 illustrates a second example quick release suture mechanism 302b, and FIGS. 10A-10D illustrate a third example quick release suture mechanism 302c. FIGS. 14A-14B illustrate a fourth example quick release suture mechanism 302d, and FIGS. 15A-15G and 15I-15M illustrate a fifth example quick release suture mechanism 302e. In general, the features, characteristics, attributes, etc. of the quick release suture mechanism 302 disclosed herein with reference to FIG. 7 also may be understood as applying to any of the quick release suture mechanisms 302a/302b/302c/302d/302e and vice-versa.

While the present disclosure generally relates to examples in which the suture lock assembly 300 and/or the quick release suture mechanism 302 is used in conjunction with a release suture 236, this is not required, and it additionally is within the scope of the present disclosure that the suture lock assembly 300 and/or the quick release suture mechanism 302 may be used in conjunction with any suitable suture. For example, the suture lock assembly 300 and/or the quick release suture mechanism 302 may be used in conjunction with a suture that is not configured to be coupled to and/or to release an implantable device. Accordingly, as used herein, the release suture 236 additionally or alternatively may be referred to as a suture 236.

Moreover, while the present disclosure generally relates to examples in which the quick release suture mechanism 302 is used in conjunction with the suture lock assembly 300, this is not required. For example, it additionally is within the scope of the present disclosure that the quick release suture mechanism 302 may be used independent of a suture lock assembly or a related suture tensioning mechanism. In particular, in some examples, the docking device delivery apparatus 50 and/or the prosthetic valve delivery apparatus 60 does not include a mechanism for adjusting a degree of slack in the release suture 236, but still may be used in conjunction with the quick release suture mechanism 302 to release the release suture 236.

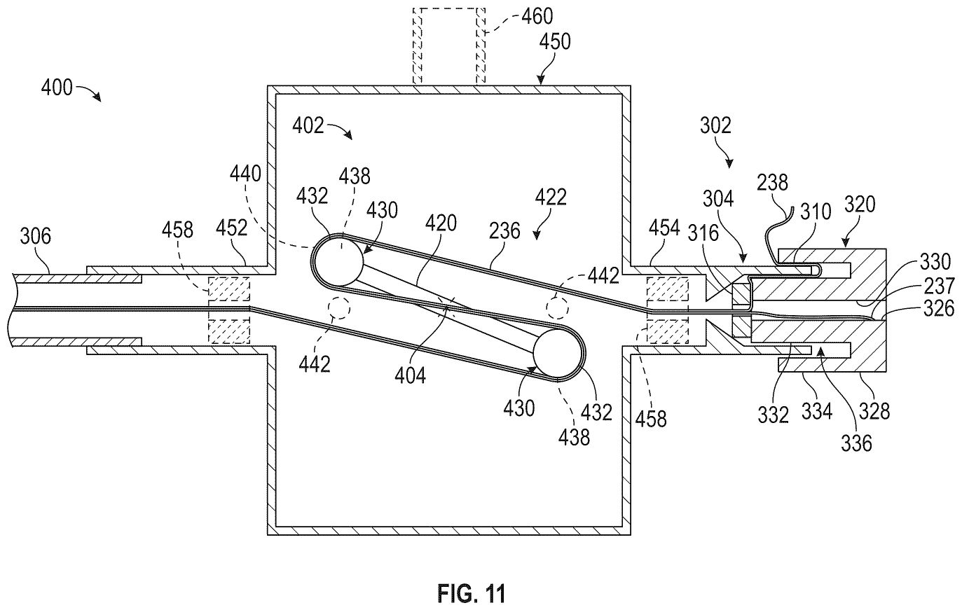

In the present disclosure, a reference numeral that includes an alphabetic label (e.g., “a,” “b,” “c,” etc.) is to be understood as labeling a particular example of the structure or component corresponding to the reference numeral. Accordingly, it is to be understood that components sharing like names and/or like reference numerals may share any properties and/or characteristics as disclosed herein even when certain such components are not specifically described and/or addressed herein. It further is to be understood that such components sharing like names and/or like reference numerals may share any properties and/or characteristics in examples in which such like reference numerals do not include an alphabetic label. As an example, the quick release suture mechanism 302 of FIG. 11, the quick release suture mechanism 302a of FIG. 8, the quick release suture mechanism 302b of FIG. 9, the quick release suture mechanism 302c of FIGS. 10A-10D, the quick release suture mechanism 302d of FIGS. 14A-14B, and/or the quick release suture mechanism 302e of FIGS. 15A-15G and 15J-15K may share any suitable properties and/or characteristics with one another. Additionally, in the present disclosure, features and/or attributes that pertain to multiple examples (e.g., examples indicated with reference numerals including respective alphabetic labels) may be presented and/or discussed with reference to a corresponding reference numeral that lacks an alphabetic label.

The tensioner housing 350 comprises a suture entry port 352 and a suture exit port 354 such that the release suture 236 extends into the tensioner housing 350 via the suture entry port 352 and out of the tensioner housing 350 via the suture exit port 354. More specifically, the release suture 236 extends between the implantable device and the spool 340 via the suture entry port 352, and the release suture 236 extends between the spool 340 and the quick release suture mechanism 302 via the suture exit port 354.

As illustrated in FIG. 7, the suture lock assembly 300 and/or the suture entry port 352 may be configured to be operatively coupled to a delivery apparatus connection 306 of the delivery apparatus 200. Accordingly, the suture lock assembly 300 may be configured such that the release suture 236 extends between the spool 340 and the implantable device via the delivery apparatus connection 306. As an example, the delivery apparatus connection 306 may represent and/or include a portion of the branch 244 of the adaptor 240 illustrated in FIG. 6B.

As illustrated in FIG. 7, the quick release suture mechanism 302 comprises a quick release dock port 304 configured to be attached to the suture exit port 354 and a suture anchor cap 320 configured to be selectively coupled to and uncoupled from the quick release dock port 304. The quick release suture mechanism 302 is configured to be selectively transitioned between a locked configuration and an unlocked configuration.

The quick release suture mechanism 302 is in the locked configuration when the suture anchor cap 320 is operatively coupled to the quick release dock port 304. In the locked configuration, the quick release suture mechanism 302 maintains the release suture 236 in a fixed position relative to the suture exit port 354 and/or relative to the quick release dock port 304, such as to restrict and/or prevent the release suture 236 from being removed from the suture lock assembly 300 or the implantable device.

The quick release suture mechanism 302 is in the unlocked configuration when the suture anchor cap 320 is removed from the quick release dock port 304. In the unlocked configuration, the release suture 236 may be removed from the suture exit port 354 and/or from the quick release dock port 304, such as to enable the release suture 236 to be removed from the implantable device.

In this manner, the quick release dock port 304 may facilitate removing the release suture 236 from the implantable device without removing the suture lock assembly 300 from the delivery apparatus connection 306 and/or without cutting the release suture. That is, in contrast with suture lock assemblies that are configured to be removed from a hub assembly in order to access and cut the release suture, removing the suture anchor cap 320 from the quick release dock port 304 offers the ability to access and remove the release suture 236 while the suture lock assembly 300 remains coupled to the delivery apparatus connection 306.

The quick release dock port 304 may be a portion of the tensioner housing 350 or may be a component that is operatively coupled to the tensioner housing 350. For example, the quick release dock port 304 and the suture exit port 354 may be integrally formed, and/or may refer to a common (e.g., the same) component.

When the quick release suture mechanism 302 is in the locked configuration, the suture lock assembly 300 may operate substantially similarly to the suture lock assembly 216 of FIGS. 6A-6D. For example, when the quick release suture mechanism 302 is in the locked configuration, rotating the spool 340 relative to the tensioner housing 350 may operate to wrap a portion of the release suture 236 around the spool 340 to increase a tension in the release suture 236 and/or to decrease a length of the release suture 236 between the suture lock assembly 300 (and/or the suture entry port 352 thereof) and the implantable device.

Unlike the example of FIGS. 6A-6D, however, the release suture 236 in the example of FIG. 7 does not terminate on the spool 340, and instead passes through each of the suture entry port 352 and the suture exit port 354. Specifically, in the example of FIG. 7, the quick release suture mechanism 302 is configured such that the release suture 236 extends from the quick release dock port 304 through the suture exit port 354 and continues through the suture entry port 352 to the implantable device and back to the quick release dock port 304 via the suture entry port 352 and the suture exit port 354.

The suture anchor cap 320 may be configured to be operatively coupled to the quick release dock port 304 in any of a variety of manners. As illustrated in FIG. 7, the quick release suture mechanism 302 may include a dock port coupling mechanism 310 that is configured to selectively couple the suture anchor cap 320 to the quick release dock port 304. In particular, one or both of the suture anchor cap 320 and/or the quick release dock port 304 may include at least a portion of the dock port coupling mechanism 310.

In some examples, such as in the examples of FIGS. 8-10D, the dock port coupling mechanism 310 comprises a port thread 312 (e.g., external threads) and a cap thread 322 (e.g., internal threads) that are configured to threadably engage one another to selectively couple the suture anchor cap 320 to the quick release dock port 304. Specifically, in such examples, the quick release dock port 304 comprises the port thread 312 and the suture anchor cap 320 comprises the cap thread 322. Accordingly, in such examples, the quick release suture mechanism 302 may be selectively transitioned from the locked configuration to the unlocked configuration by unscrewing the suture anchor cap 320 from the quick release dock port 304. The pitch of the port thread 312 and/or the cap thread 322 can be configured to allow the suture anchor cap 320 to be removed from the quick release dock port 304 via a desired number of revolutions of the suture anchor cap 320 relative to the quick release dock port 304 (e.g., less than one revolution, one revolution, or more than one revolution). While FIGS. 8-10D illustrate the port threads 312 as being external threads and the cap threads 322 as being internal threads, it also is within the scope of the present disclosure that the port threads 312 can be internal threads and that the cap threads 322 can be external threads.

In other examples, such as in the example of FIGS. 15A-15G and 15I-15M, the dock port coupling mechanism 310 may include and/or be a bayonet lock coupling mechanism. Specifically, in the example of FIGS. 15A-15G and 15I-15M, the dock port coupling mechanism 310e comprises a bayonet pin 314e and a bayonet slot 324e configured to receive the bayonet pin 314e in a bayonet lock configuration. In the example of FIGS. 15A-15G and 15I-15M, the quick release dock port 304e comprises the bayonet pin 314e and the suture anchor cap 320e comprises the bayonet slot 324e. In other examples, however, the quick release dock port 304 may comprise the bayonet slot 324 and the suture anchor cap 320 may comprise the bayonet pin 314. The bayonet type coupling mechanism can, for example, allow the suture anchor cap 320 to be released with less than one revolution of the suture anchor cap 320 relative to the quick release dock port 304 (e.g., ¼ turn, ½ turn, etc.).

Accordingly, in such examples, the quick release suture mechanism 302 may be selectively transitioned from the locked configuration to the unlocked configuration by sequentially rotating the suture anchor cap 320 relative to the quick release dock port 304 and axially translating the suture anchor cap 320 away from the quick release dock port 304.

Such a two-stage removal mechanism may protect against inadvertently removing the suture anchor cap 320 from the quick release dock port 304. Additional or alternative examples of mechanisms for mitigating the likelihood of inadvertently removing the suture anchor cap 320 from the quick release dock port 304 may include two-stage removal mechanisms (e.g., grenade pin mechanisms, locking ratchets, removable physical locks, etc.), physical barriers, visual indicators, etc.

In some examples in which the dock port coupling mechanism 310 includes the bayonet lock coupling mechanism, the dock port coupling mechanism 310 includes one or more features to retain the suture anchor cap 320 in engagement with the quick release dock port 304. For example, and as discussed in more detail below, the dock port coupling mechanism 310e can include one or more features to urge the suture anchor cap 320e axially outward relative to the quick release dock port 304e, such as to ensure that the bayonet pin 314e remains engaged with the bayonet slot 324e.

In some examples, and as illustrated at least in FIGS. 10A-10C, 15A-15G, and 15I-15M, the suture anchor cap 320 comprises a grip feature 328 configured to facilitate gripping the suture anchor cap 320 to transition the quick release suture mechanism 302 between the locked configuration and the unlocked configuration. The grip feature 328 may include and/or be any suitable feature, examples of which include a textured surface, an indentation, a protrusion, a knob, a lever, a handle, a tab, etc.

In particular, FIGS. 10A-10C illustrate an example in which the grip feature 328c comprises a set of raised ridges to facilitate unscrewing the suture anchor cap 320 from the quick release dock port 304. In the example of FIGS. 15A-15G and 15I-15M, the grip feature 328e comprises a tab configured to facilitate actuating the bayonet lock mechanism of the dock port coupling mechanism 310e.

The suture anchor cap 320 may have any of a variety of structures and/or configurations. In some examples, and as illustrated in FIG. 7, the suture anchor cap 320 comprises an inner plug 332 that is at least partially received within the quick release dock port 304 when the quick release suture mechanism 302 is in the locked configuration. The suture anchor cap 320 additionally may comprise an outer skirt 334 that circumferentially surrounds the inner plug 332 and that extends circumferentially around the quick release dock port 304 when the quick release suture mechanism 302 is in the locked configuration. In such examples, the suture anchor cap 320 further may comprise an annular channel 336 that is defined between the inner plug 332 and the outer skirt 334, such that the annular channel 336 receives at least a portion of the quick release dock port 304 when the quick release suture mechanism 302 is in the locked configuration.

In an example in which the dock port coupling mechanism 310 comprises a threaded coupling, and as illustrated in FIGS. 8-9, an inner surface of the outer skirt 334 may comprise and/or define the cap thread 322, and an outer surface of the quick release dock port 304 may comprise and/or define the port thread 312. In other examples, the inner plug 332 may comprise and/or define the cap thread 322, and/or an inner surface of the quick release dock port 304 may comprise and/or define the port thread 312.

The suture anchor cap 320 may be configured to be operatively coupled to the release suture 236 in any of a variety of manners. In particular, in various examples, the release suture 236 extends between and terminates at a suture anchored end 237 and a suture free end 238, and the suture anchor cap 320 comprises a suture anchor location 326 at which the suture anchored end 237 is configured to be coupled. Specifically, the suture anchor cap 320 is configured to be operatively coupled to the suture anchored end 237 at the suture anchor location 326 such that the suture anchored end 237 is restricted from being removed from the suture anchor cap 320 during operative use of the quick release suture mechanism 302 (e.g., when the quick release suture mechanism 302 is in either of the locked configuration or the unlocked configuration).

In some examples, the suture anchored end 237 is fixedly coupled to the suture anchor location 326, such as via an adhesive, a cement, a mechanical coupling, a tied coupling, a mechanical obstruction (e.g., between a knot in the release suture 236 and a smaller aperture in the suture anchor cap 320), and/or other means for fixedly coupling the suture anchored end 237 to the suture anchor location 326.

As used herein, the suture anchored end 237 and/or the suture free end 238 may refer to respective terminal ends of the release suture 236 or may refer to respective portions of the release suture 236 that extend proximate to these terminal ends. Thus, for example, the suture anchored end 237 and/or the suture free end 238 may be described as being attached to another component when a region of the release suture 236 proximate to such a terminal end is attached to the other component.

The quick release suture mechanism 302 generally is configured such that the suture free end 238 is fixed in position when the quick release suture mechanism 302 is in the locked configuration. In particular, when the quick release suture mechanism 302 is in the locked configuration, the suture free end 238 is at least substantially fixed in position relative to the quick release dock port 304. Such a configuration may operate to prevent the release suture 236 from being removed from the quick release suture mechanism 302 or from the implantable device prior to transitioning the quick release suture mechanism 302 to the unlocked configuration.

Alternatively, when the quick release suture mechanism 302 is in the unlocked configuration, the suture free end 238 is free to move through the quick release dock port 304. Thus, when the quick release suture mechanism 302 is in the unlocked configuration, the release suture 236 may be removed from the implantable device by withdrawing an entirety of the release suture 236 from the suture lock assembly 300, form the delivery apparatus 100, and/or from the implantable device.

The quick release suture mechanism 302 may be configured to engage the suture free end 238 in any suitable manner when the quick release suture mechanism 302 is in the locked configuration. For example, and as illustrated in FIG. 7, when the quick release suture mechanism 302 is in the locked configuration, the suture free end 238 may be interposed between the quick release dock port 304 and the suture anchor cap 320. As more specific examples, the suture free end 238 may be pinched between the quick release dock port 304 and the inner plug 332 and/or between the quick release dock port 304 and the outer skirt 334.

As a more specific example, such as in the example of FIG. 8, the quick release suture mechanism 302 is configured such that, when the quick release suture mechanism 302 is in the locked configuration, the suture free end 238 extends between the port thread 312 and the cap thread 322. Accordingly, in such examples, the suture free end 238 may be locked in position via the threaded engagement between the port thread 312 and the cap thread 322.

In some examples (see, e.g., FIGS. 7-10D), the suture anchor cap 320 defines an inner bore 330 that extends at least partially through the suture anchor cap 320. In particular, FIGS. 7-9 illustrate examples in which the inner bore 330 extends fully through a length of the suture anchor cap 320, while FIGS. 10A-10D illustrate an example in which the inner bore 330 extends only partially through the suture anchor cap 320. In some examples, the inner plug 332 defines at least a portion of the inner bore 330.

In some examples, the suture anchored end 237 and/or the suture free end 238 may extend at least partially through the inner bore 330. In some examples (see, e.g., FIGS. 7-9), the inner bore 330 may comprise the suture anchor location 326. For example, the suture anchored end 237 may be fixedly coupled to the suture anchor cap 320 (e.g., via an adhesive, a cement, and/or any other suitable bonding means) at a location on an inner surface of the suture anchor cap 320 that defines the inner bore 330. Additionally or alternatively, and as described in more detail below, the inner bore 330 may include one or more geometrical features (e.g., a constriction, a bend, etc.) that serves as the suture anchor location 326 by at least partially restricting the release suture 236 from being removed from the inner bore 330.

In some examples, and as illustrated in FIGS. 7-8, the quick release suture mechanism 302 is configured such that the suture free end 238 extends at least substantially exterior of the inner bore 330 when the quick release suture mechanism 302 is in the locked configuration. In particular, in some such examples, the suture free end 238 extends from the suture exit port 354 through the quick release dock port 304, around an exterior surface of the inner plug 332 and through the annular channel 336 such that the suture free end 238 extends away from the interface between the suture anchor cap 320 and the quick release dock port 304. Thus, in such examples, although a portion of the suture free end 238 may enter the inner bore 330, the suture free end 238 may not be configured to be accessed via the inner bore 330 when the quick release suture mechanism 302 is in the locked configuration.

In other examples, the quick release suture mechanism 302 may be configured such that the suture free end 238 extends at least partially through the inner bore 330 when the quick release suture mechanism 302 is in the locked configuration. For example, and as illustrated in FIG. 9, the suture free end 238 may extend from the suture exit port 354 through the inner bore 330, around the outer skirt 334, into the annular channel 336 around an end of the quick release dock port 304, and again through the inner bore 330.

Alternatively, the suture free end 238 may extend from the suture exit port 354 and around the end of the quick release dock port 304 via the annular channel 336, around the outer skirt 334, and through the inner bore 330.

In each such example, and similar to the example of FIG. 8, the suture free end 238 may be trapped between the port thread 312 and the cap thread 322 when the quick release suture mechanism 302 is in the locked configuration.

In some examples, the suture anchor cap 320 may define a plurality of inner bores 330. For example, and as illustrated in FIG. 15I-15K, the inner bore 330 may be a first inner bore 330, and the suture anchor cap 320 further may comprise a second inner bore 331. In such examples, the suture anchored end 237 may extend at least partially through the first inner bore 330, and/or the suture free end 238 may extend at least partially through the second inner bore 331 (e.g., when the quick release suture mechanism is in the locked configuration).

Each of the suture anchored end 237 and the suture free end 238 may be operatively coupled to the suture anchor cap 320 in any of a variety of manners. For example, and as illustrated in FIG. 15J, the suture anchored end 237 may extend through the first inner bore 330e and terminate in a knot. In this manner, the knotted end of the suture anchored end 237 may fit within the recess at the suture anchor location 326e but may be too large to pass through the first inner bore 330e.

As another example, and as illustrated in FIG. 15K, the suture anchored end 237 may extend through the first inner bore 330e and continue around an exterior of the inner plug 332e to be tied and/or otherwise affixed to a portion of the release suture 236 to secure the suture anchored end 237 to the suture anchor cap 320e.

In other examples, the suture anchored end 237 additionally or alternatively may be affixed (e.g., with an adhesive, with a screw, etc.) to the suture anchor location 326, to the first inner bore 330, and/or to the suture anchor cap 320.

The suture free end 238 also may be fixed in position relative to the suture anchor cap 320 in any of a variety of manners. In the example of FIGS. 15A-15M, and as shown in FIGS. 15E and 15J-15M, the dock port coupling mechanism 310e comprises a pair of bayonet pins 314e, at least one of which terminates at a pin cap 315e that is wider in diameter than a remainder of the bayonet pin 314e. In some examples, such as in the examples of FIGS. 15J-15M, the suture free end 238 may be wrapped around the bayonet pin 314e such that the pin cap 315e restricts the suture free end 238 from falling off of an end of the bayonet pin 314e. Stated differently, in such examples, the pin cap 315e may operate to retain the release suture 236 on the bayonet pin 314e. This is not required of all examples, however, and it additionally is within the scope of the present disclosure that the suture free end 238 may be sufficiently retained on the bayonet pin 314e in the absence of the pin cap 315e and/or without engaging the pin cap 315e.

Additionally, or alternatively, and as illustrated in FIGS. 15E and 15J-15M, at least one bayonet pin 314e may comprise a transverse pin bore 317e extending through a diameter of the bayonet pin 314e. In such examples, and as shown in FIGS. 15J-15K, the release suture 236 and/or the suture free end 238 thereof may extend through the transverse pin bore 317e to at least partially retain the suture free end 238 in position relative to the bayonet pin 314e.

Additionally, or alternatively, in some examples, the suture free end 238 can be wrapped around each of the pair of bayonet pins 314e. For example, the suture free end 238 can be wrapped around a first bayonet pin 314e, wrapped around an end of the quick release dock port 304e, and wrapped around a second bayonet pin 314e. In such examples, the suture free end 238 can be wrapped in a figure-8 pattern around the quick release dock port 304e and the bayonet pins 314e. It further is within the scope of the present disclosure that any other suitable patterns or configurations may be used to wrap the suture free end 238 around one or more components of the quick release suture mechanism 302, such as to achieve a desired suture retention strength.

In some examples, and as illustrated in FIGS. 15J-15L, when the quick release suture mechanism 302e is in the locked configuration, the suture free end 238 of the release suture 236 may extend through the second inner bore 331e and through the annular channel 336e to be wrapped around the bayonet pin 314e. In some such examples, the engagement between the suture free end 238 and one or both of the bayonet pin 314e and the bayonet slot 324e may be sufficient to maintain the suture free end 238 in position while the quick release suture mechanism 302e is in the locked configuration.

In other examples, and as illustrated in FIG. 15M, when the quick release suture mechanism 302e is in the locked configuration, the suture free end 238 of the release suture 236 may extend through the inner bore 331e and through the annular channel 336e (as shown in FIGS. 15J-15K) and may extend exterior of the outer skirt 334e to loop around the outer skirt 334e and continue back to the bayonet pin 314e. The suture free end 238 further may extend through the annular channel 336e between the outer skirt 334e and the quick release dock port 304e. In this manner, the suture free end 238 may be pinched between the outer skirt 334e and the quick release dock port 304e, and/or may be tensioned between the edge of the outer skirt 334e and the bayonet pin 314e, to further secure the suture free end 238 relative to the bayonet pin 314e.

In some examples, the suture free end 238 additionally or alternatively may be fixedly coupled to the suture anchor cap 320 at least when the quick release suture mechanism 302 is in the locked configuration, and/or may remain attached to the suture anchor cap 320 when the suture anchor cap 320 is removed from the quick release dock port 304. In such examples, the release suture 236 may be removed from the suture lock assembly 300/400 and/or the delivery apparatus 200 by severing the release suture 236 subsequent to removing the suture anchor cap 320 from the quick release dock port 304. Accordingly, in such examples, the unlocked configuration of the quick release suture mechanism 302 may correspond to a configuration in which the suture anchor cap 320 is removed from the quick release dock port 304 and in which the suture free end 238 subsequently is removed from the suture anchor cap 320.

In other examples, the quick release suture mechanism 302e of FIGS. 15A-15I may be used such that the suture free end 238 does not extend through the second inner bore 331e and/or is not directly coupled to the suture anchor cap 320e as described above. For example, the suture free end 238 additionally or alternatively may be coupled to (e.g., wrapped around) the bayonet pin 314e of the quick release dock port 304e, at least when the quick release suture mechanism 302e is in the locked configuration. Accordingly, in such examples, the quick release suture mechanism 302e may be transitioned from the locked configuration to the unlocked configuration by first uncoupling (e.g., unwrapping) the suture free end 238 from the bayonet pin 314e.

In some examples, and as illustrated in FIG. 7, the quick release suture mechanism 302 comprises a gasket 316 that is configured to form an at least substantially fluid-tight seal to restrict leakage out of the quick release dock port 304. In particular, in some examples, the gasket 316 is configured to form a substantially fluid-tight seal between the suture anchor cap 320 and the quick release dock port 304 when the quick release suture mechanism 302 is in the locked configuration. In some examples, the gasket 316 may be positioned within the suture exit port 354 and/or within the quick release dock port 304.

Additionally, or alternatively, in some examples, the gasket 316 may be configured to restrict fluid from exiting the quick release dock port 304 when the quick release suture mechanism 302 is in the unlocked configuration. That is, when the suture anchor cap 320 is removed from the quick release dock port 304, the quick release dock port 304 may form a leak path out of the delivery apparatus 200. To mitigate this, the gasket 316 may operate to restrict and/or prevent blood, saline, and/or other fluid from exiting the quick release dock port 304 when the suture anchor cap 320 is removed.

In some examples, the gasket 316 may include and/or be a gasket that opens when compressed, such as a gasket commonly used in needleless valves. In such an example, the gasket 316 may be open to a fluid flow when the suture anchor cap 320 is operatively coupled to the quick release dock port 304, and the gasket 316 may close to a fluid flow when the suture anchor cap 320 is removed from the quick release dock port 304.

As another example, the gasket 316 may include and/or be a Tuohy Borst gasket, which compresses and closes when the suture anchor cap 320 is operatively coupled to the quick release dock port 304, and which opens slightly when the suture anchor cap 320 is removed from the quick release dock port 304. In such an example, the slight opening of the gasket 316 may facilitate pulling the release suture 236 through the gasket 316.

In an example in which the dock port coupling mechanism 310 comprises the bayonet pin 314 and the bayonet slot 324, the gasket 316 additionally may operate to assist in retaining the quick release suture mechanism 302 in the locked configuration. For example, when the quick release suture mechanism 302e is in the locked configuration, and as shown in FIG. 15E, the suture anchor cap 320e may slightly compress the gasket 316e such that the gasket 316e urges the suture anchor cap 320e axially outward, thus urging the bayonet pin 314e into engagement with the bayonet slot 324e. Stated differently, when the quick release suture mechanism 302e is in the locked configuration, the gasket 316e can urge the suture anchor cap 320e axially outward to maintain the engagement between the bayonet pin 314e and the bayonet slot 324e. In this manner, the gasket 316e also may introduce a two-stage removal mechanism to the quick release suture mechanism 302e by requiring that a user push the suture anchor cap 320e axially inward against the outward force of the gasket 316e prior to rotating the suture anchor cap 320e relative to the quick release dock port 304e.

Additionally, and as shown in FIGS. 15J-15M, in an example in which the suture free end 238 of the release suture 236 wraps around the bayonet pin 314e, the action of the gasket 316e to urge the bayonet pin 314e into engagement with the bayonet slot 324e also may serve to pinch the release suture 236 between the bayonet pin 314e and the bayonet slot 324e. In this manner, the action of the gasket 316e to urge the bayonet pin 314e into engagement with the bayonet slot 324e may serve to secure the suture free end 238 of the release suture 236 in position while the quick release suture mechanism 302e is in the locked configuration.

Additionally, or alternatively, in some examples, and as illustrated in FIGS. 15L-15M, a portion of the bayonet slot 324e may extend toward the quick release dock port 304e (e.g., when the quick release suture mechanism 302e is in the locked configuration) to define a recess for receiving the bayonet pin 314e. Accordingly, in such examples, the suture anchor cap 320e may need to be pushed axially inward against the gasket 316e before rotating and subsequently removing the suture anchor cap 320e from the quick release dock port 304e.

In some examples, and as illustrated in FIG. 7, the quick release suture mechanism 302 additionally comprises one or more suture centralizers 358 configured to guide the release suture 236 through the tensioner housing 350. For example, when present, each suture centralizer may be configured to engage the tensioner housing 350 (e.g., within the suture entry port 352 or within the suture exit port 354) and to support the release suture 236 through a central bore or passage thereof to maintain at least a portion of the release suture 236 away from an interior surface of the tensioner housing 350. In some examples, the gasket 316 may operate as a suture centralizer 358.

As further illustrated in FIG. 7, the suture lock assembly 300 additionally may include a flush port 360 fluidly coupled to the suture entry port 352 for supplying a flush fluid into the tensioner housing 350 and/or to a downstream portion of the delivery apparatus 200. In some such examples, the tensioner housing 350 at least partially defines the flush port 360. In particular, because the quick release suture mechanism 302 enables the release suture 236 to be removed from the implantable device without removing the suture lock assembly 300 from the delivery apparatus connection 306, the flush fluid may be delivered to the delivery apparatus 200 via the tensioner housing 350 without a loss of hemostasis.

When present, the flush port 360 may include and/or be any suitable port and/or coupling, such as a port that nominally is sealed and that opens when another component (e.g., a flush fluid source) is operatively coupled to the flush port 360. In some examples, the flush port 360 may include and/or be a Luer lock port and/or a swabbable Luer lock port.

FIGS. 10A-10D sequentially illustrate an exemplary process by which the release suture 236 may be withdrawn from the quick release suture mechanism 302c. In particular, FIG. 10A illustrates the suture anchor cap 320c operatively coupled to the quick release dock port 304c such that the quick release suture mechanism 302c is in the locked configuration.

FIG. 10B illustrates the suture anchor cap 320c removed from the quick release dock port 304c such that the quick release suture mechanism 302c is in the unlocked configuration. As shown in FIG. 10B, the suture anchored end 237 is fixedly coupled to the suture anchor cap 320c inside the inner bore 330, while the suture free end 238 extends out of the quick release dock port 304c.

With the suture free end 238 freed from the interface between the suture anchor cap 320c and the quick release dock port 304c, pulling the suture anchor cap 320c away from the quick release dock port 304c (e.g., to the configuration of FIG. 10C) pulls the suture free end 238 into the quick release dock port 304c and to the implantable device. As the suture anchor cap 320c is pulled further, the suture free end 238 may be fully withdrawn from the implantable device and the quick release dock port 304c, as shown in FIG. 10D.

While the foregoing descriptions of the suture anchor cap 320 generally relate to examples in which the suture lock assembly 216/300 comprises a spool 278/340 to apply tension to the release suture 236, this is not required of all examples. For example, FIG. 11 illustrates examples of a suture lock assembly 400 that comprises a suture tensioner 402 to engage and selectively apply tension to a release suture 236 that is attached to an implantable device (such as the docking device 70). The suture anchor caps 320 also can be used with other delivery assemblies in which an implantable device is releasably coupled to a delivery apparatus with one or more sutures. For example, and as discussed above, the suture anchor caps 320 can be used in conjunction with a docking device delivery apparatus 50 and/or a prosthetic valve delivery apparatus 60 that lacks a mechanism for adjusting a degree of slack in the release suture 236.

The suture lock assembly 400 may include any features, characteristics, attributes, etc. disclosed herein with reference to the suture lock assembly 216 and/or the suture lock assembly 300, and vice versa. In particular, FIG. 11 illustrates an example in which the suture lock assembly 400 comprises the quick release suture mechanism 302 discussed above with reference to FIGS. 7-10D.

While the present disclosure generally relates to examples in which the suture lock assembly 400 is used in conjunction with a release suture 236, this is not required, and it additionally is within the scope of the present disclosure that the suture lock assembly 400 may be used in conjunction with any suitable suture. For example, the suture lock assembly 400 may be used in conjunction with a suture that is not configured to be coupled to and/or to release an implantable device. Accordingly, as used herein, the release suture 236 additionally or alternatively may be referred to as a suture 236.

As illustrated in FIG. 11, the suture lock assembly 400 comprises a tensioner housing 450 that at least partially receives the suture tensioner 402. The tensioner housing 450 comprises a suture entry port 452 such that the release suture 236 extends between the implantable device and the suture tensioner 402 via the suture entry port 452. In some examples, and as illustrated in FIG. 11, the tensioner housing 450 additionally comprises a suture exit port 454 such that the release suture 236 extends through the tensioner housing 450 between the suture entry port 452 and the suture exit port 454.

As illustrated in FIG. 11, the suture lock assembly 400 and/or the suture entry port 452 may be configured to be operatively coupled to a delivery apparatus connection 306 of the delivery apparatus 200. Accordingly, the suture lock assembly 400 may be configured such that the release suture 236 extends between the suture tensioner 402 and the implantable device via the delivery apparatus connection 306. As an example, the delivery apparatus connection 306 may represent a portion of the branch 244 of the adaptor 240 illustrated in FIG. 6B.

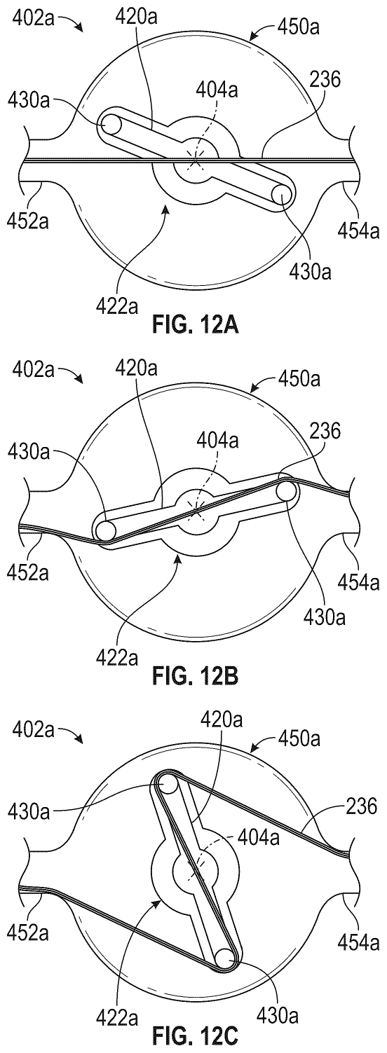

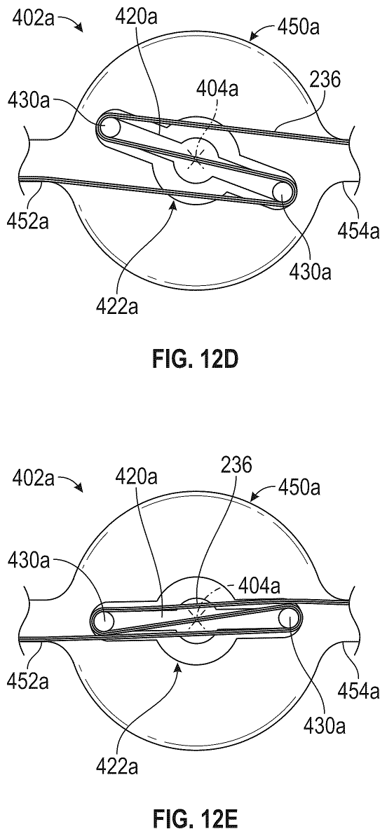

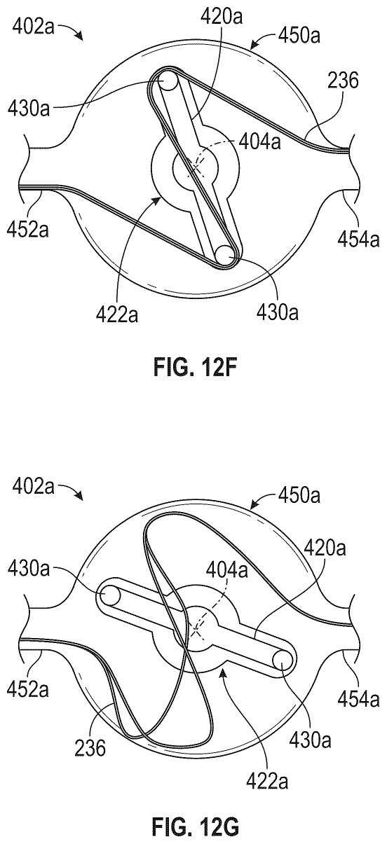

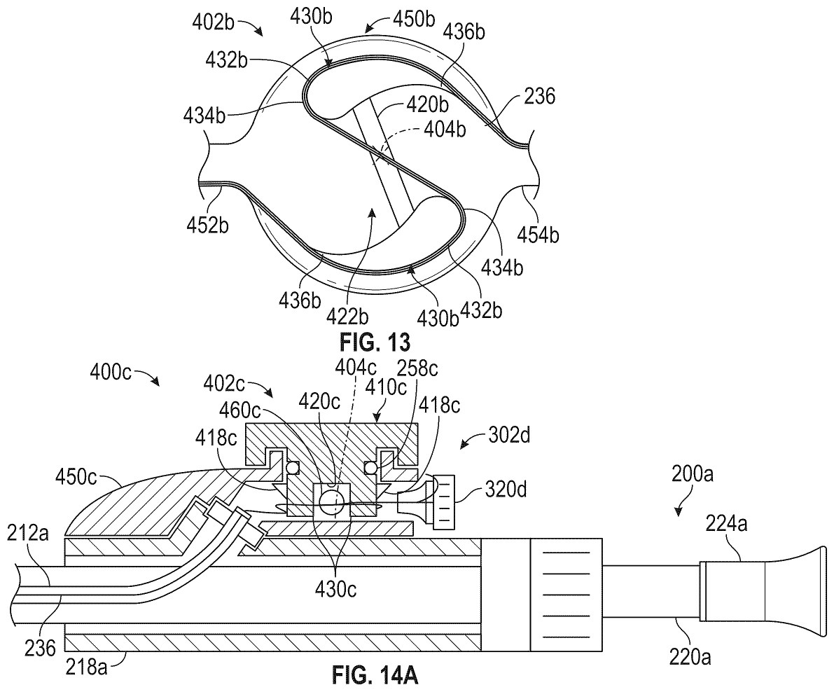

FIGS. 12A-15M illustrate more specific examples of suture lock assemblies 400 and/or of components thereof, as described in more detail below. In particular, FIGS. 12A-12G illustrate a first example suture lock assembly 400a, while FIG. 13 illustrates a second example suture lock assembly 400b. FIGS. 14A-14B illustrate a third example suture lock assembly 400c that incorporates the fourth example quick release suture mechanism 302d, and FIGS. 15A-15M illustrate a fourth example suture lock assembly 400d that incorporates the fifth example quick release suture mechanism 302e. In general, the features, characteristics, attributes, etc. of the suture lock assembly 400 disclosed herein with reference to FIG. 11 also may be understood as applying to any of the suture lock assemblies 400a/400b/400c/400d and vice-versa.

Returning to FIG. 11, the suture tensioner 402 of the suture lock assembly 400 is configured to be selectively rotated relative to the tensioner housing 450 about a tensioner central axis 404. Specifically, the suture tensioner 402 includes a pair of spaced-apart posts 430 that are coupled to one another via a connecting body 420 (e.g., an arm, a hub, a disc, a wheel, etc.) such that the posts 430 are configured to revolve in unison around the tensioner central axis 404 as the suture tensioner 402 is rotated. In some examples, the posts 430 and/or the connecting body 420 collectively may be referred to as a forked tensioner mechanism.

As the suture tensioner 402 is rotated relative to the tensioner housing 450 in a first direction (e.g., counterclockwise in the example of FIG. 11), the posts 430 engage and collect (e.g., wrap up) a length of the release suture 236 to selectively increase a length of the release suture 236 that is enclosed by the tensioner housing 450, thereby selectively increasing a tension in the release suture 236. In some examples, the suture lock assembly 400 may be configured such that the release suture 236 extends from the suture tensioner 402 through the suture entry port 452 to the implantable device and back to the suture tensioner 402 via the suture entry port 452. Accordingly, in such examples, rotating the suture tensioner 402 in the first direction to increase the length of the release suture 236 contained within the tensioner housing 450 operates to increase a tension in the release suture 236 between the suture tensioner 402 and the implantable device.

With the release suture 236 in a tensioned state after rotating the suture tensioner 402 in the first direction, rotating the suture tensioner 402 in a second direction opposite the first direction (e.g., clockwise in the example of FIG. 11) operates to progressively release the tension in the release suture 236. As the suture tensioner 402 is rotated in the second direction, the release of tension may allow slack to form in the release suture 236. For example, if the tension exerted by the suture tensioner 402 is released, and if no external tension force operates to pull the release suture 236 out of the tensioner housing 450 via the suture entry port 452, then the slackened release suture 236 may remain within the tensioner housing 450. As discussed in more detail below, the spaced-apart configuration of the posts 430 may enable the slackened release suture 236 to occupy the tensioner housing 450 and/or to reassume a tensioned configuration with minimal risk of tangling.

In some examples, as illustrated in FIG. 11 and as discussed in more detail below, the suture lock assembly 400 and/or the suture tensioner 402 is configured to be utilized in conjunction with the quick release suture mechanism 302 and/or the suture anchor cap 320 disclosed herein. In particular, in some such examples, the quick release suture mechanism 302 is coupled to and/or incorporated with the suture exit port 454. In such examples, the suture lock assembly 400 may be described as being similar and/or equivalent to an example of the suture lock assembly 300 in which the spool 340 is replaced with the suture tensioner 402 as described herein.

In other examples, however, the suture lock assembly 400 and/or the suture tensioner 402 may not be configured to be utilized with the quick release suture mechanism 302 and/or the suture anchor cap 320. In particular, in some examples, the tensioner housing 450 may lack the suture exit port 454, and/or a portion of the release suture 236 may be fixedly coupled to one of the posts 430. For example, the release suture 236 may extend between and terminate at a first end and a second end, at least one of which is fixedly coupled to a respective suture anchor location 440 of the suture tensioner 402. As illustrated in FIG. 11, at least one of the posts 430 may include and/or define such a suture anchor location 440.

Each of the posts 430 may have any suitable shape and/or configuration for engaging the release suture 236 as described herein. In some examples, and as illustrated in FIGS. 11-12G, 14A, and 15D-15H, each post 430 is substantially cylindrical, with a circular cross-sectional shape.

This is not required of all examples, however, and it additionally is within the scope of the present disclosure that each post 430 may have any of a variety of shapes. As an example, as illustrated in FIG. 13 and as described in more detail below, each post 430 may have an asymmetric shape, such as a yin-yang shape.

As additional examples, each post 430 may have a shape that is conical and/or frusto-conical, and/or may have a cross-sectional shape that is curved, circular, oval, and/or elliptical.

Additionally, while the present disclosure generally relates to examples in which the posts 430 are at least substantially identical to one another (e.g., in shape and/or size), it also is within the scope of the present disclosure that the pair of posts 430 may differ in shape and/or size.

FIG. 11 illustrates a configuration in which the suture tensioner 402 applies tension to the release suture 236, while FIGS. 12A-12G illustrate a sequence of events in which the suture tensioner 402a applies increasing tension to the release suture 236 and subsequently releases this tension.

The suture tensioner 402 may be described as transitioning among a plurality of tensioner configurations to selectively apply tension to the release suture 236. For example, rotating the suture tensioner 402 relative to the tensioner housing 450 may be described as operating to transition the suture tensioner 402 among a plurality of tensioner configurations defined between and comprising a released configuration and a fully tensioned configuration.

In the released configuration, the posts 430 are positioned to exert no (or negligible) force on the release suture 236. For example, when the suture tensioner 402 is in the released configuration, and as shown in FIG. 12A, one or both of the posts 430 may be spaced apart from a straight line connecting the suture entry port 452 and the suture exit port 454 or may be tangent to such a straight line. In particular, FIG. 12A illustrates a configuration in which the release suture 236 extends between the suture entry port 452a and the suture exit port 454a and in which each post 430 is spaced apart from the release suture 236.

In various examples, any of the plurality of rotational orientations of the suture tensioner 402 relative to the tensioner housing 450 in which the posts 430 are spaced apart from such a straight line connecting the suture entry port 452 and the suture exit port 454 may be described as representing the released configuration.

Stepping sequentially through the configurations illustrated in FIGS. 12A-12E, it may be seen that rotating the suture tensioner 402a relative to the tensioner housing 450a (counterclockwise in this example) operates to bring the posts 430a into engagement with the release suture 236. As the suture tensioner 402a continues to rotate, the posts 430a wrap up an increasing length of the release suture 236 such that a length of the release suture 236 extending between the suture entry port 452a and the suture exit port 454a increases. This may operate to increase the tension in the release suture 236, such as when a distal portion of the release suture 236 is coupled to an implantable device that is fixed in position axially relative to the suture lock assembly 400. FIG. 12E represents the fully tensioned configuration of the suture tensioner 402a, in which the posts 430a are positioned to engage the release suture to yield a maximum tension in the release suture 236 when the release suture 236 is coupled to the implantable device.

FIGS. 12E-12G illustrate a sequence of events in which, starting from the fully tensioned configuration, the suture tensioner 402a is rotated relative to the tensioner housing 450a (clockwise in this example) to decrease a tension in the release suture 236. In particular, when a distal end of the release suture 236 is coupled to an implantable device that experiences no distally directed force, rotating the suture tensioner 402a from the fully tensioned configuration of FIG. 12E toward the released configuration of FIG. 12G allows slack to develop in the release suture 236.

As shown in FIGS. 12F-12G, because the posts 430a that operate to tension the release suture 236 are spaced apart from one another, the space between the posts 430a may allow the release suture 236 to release its tension with a low risk of tangling. Specifically, because the release suture 236 does not experience a distally directed force (i.e., directed out of the tensioner housing 450a via the suture entry port 452a) in this example, the length of the release suture 236 contained within the tensioner housing 450a will remain substantially unchanged as the tension exerted by the suture tensioner 402a is released.

As illustrated in FIG. 12G, the spaced-apart posts 430a may afford the release suture 236 ample volume to spread out without tangling with itself. In particular, and as illustrated in FIGS. 11-13, the tensioner housing 450 may comprise and/or define a tensioner chamber 422 that encloses at least a portion of the posts 430 and a portion of the release suture 236. Specifically, when the suture tensioner 402 is in the released configuration, the release suture 236 is free to occupy a region of the tensioner chamber 422 between the posts 430 without obstruction by the suture tensioner 402. Stated differently, the suture lock assembly 400 may be configured such that the suture tensioner 402 may be rotated to a position (e.g., the released configuration) in which the posts 430 are spaced apart from the release suture 236.

In contrast with a suture lock assembly that uses a spool to apply tension to the release suture, the spaced-apart posts 430 of the suture lock assembly 400 may offer a greater capacity to increase the length of the release suture 236 within the tensioner housing 450 per rotation of the suture tensioner 402. For example, rotating a spool (e.g., the spool 278 or the spool 340) through half a rotation will collect a length of the release suture 236 that is approximately equal to half the circumference of the spool. By contrast, and as may be seen comparing FIGS. 12A and 12E (discussed in more detail below), rotating the suture tensioner 402 through half a rotation may collect a length of the release suture 236 that is approximately equal to twice the distance separating the posts 430.

Additionally, the spaced-apart posts 430 of the suture tensioner 402 may offer enhanced stability and/or security of the release suture 236 when the suture tensioner 402 is in the fully tensioned configuration relative to a suture lock assembly that utilizes a spool. For example, and with reference to the suture lock assembly 300 of FIG. 7, when the spool 340 operates to exert a tension on the release suture 236, the release suture 236 will impart a torque on the spool 340 with a magnitude equal to the product of the tension in the release suture 236 and the radius of the spool 340. By contrast, and with reference to FIG. 12E, when the suture tensioner 402a is in the fully tensioned configuration, the release suture 236 may be at least substantially aligned with the posts 430a such that the release suture 236 exerts minimal torque upon the suture tensioner 402a. Accordingly, less force and/or mechanical resistance may be needed to maintain the suture tensioner 402a in the fully tensioned configuration (e.g., to counteract the pull of the release suture 236) relative to a configuration that utilizes a spool (e.g., the spool 278 or the spool 340) to apply tension to the release suture 236.

As discussed above with reference to FIGS. 12A and 12G, the suture tensioner 402 may be described as being in the released configuration when the suture tensioner 402 is in any of a variety of rotational configurations in which the posts 430 do not obstruct and/or exert a tension upon the release suture 236. For example, and as shown in FIG. 12G, the spaced-apart posts 430a may be positioned in any of a variety of rotational configurations corresponding to the released configuration while affording.

Similarly, the fully tensioned configuration of the suture tensioner 402 may refer to any rotational configuration of the suture tensioner 402 and/or of the posts 430 relative to the tensioner housing 450 in which the posts 430 maximize the length of the release suture 236 extending within the tensioner housing 450.

In the example of FIGS. 12A-12G, the suture tensioner 402 rotates relative to the tensioner housing 450 through an angle that is about 180 degrees to transition the suture tensioner 402 between the released configuration and the fully tensioned configuration. This is not required of all examples, however, and it additionally is within the scope of the present disclosure that the suture tensioner 402 may rotate through any suitable angle to transition between the released configuration and the fully tensioned configuration. As examples, the suture tensioner 402 may be configured to rotate relative to the tensioner housing 450 through an angle that is less than 180 degrees, about 180 degrees, more than 180 degrees, less than 360 degrees, about 360 degrees, and/or more than 360 degrees.

In some examples, the suture lock assembly 400 may include one or more features to mechanically limit a rotational range of motion of the suture tensioner 402 and/or to define the released configuration and/or the fully tensioned configuration. For example, and as illustrated in FIG. 11, the suture lock assembly 400 may comprise one or more rotator stops 442 that are fixed in position relative to the tensioner housing 450. In some examples, at least one rotator stop 442 extends into the tensioner chamber 422 and is configured to engage one or both of the posts 430 when the suture tensioner 402 is in the released configuration and/or in the fully tensioned configuration to define the released configuration and/or the fully tensioned configuration.

Additionally, or alternatively, at least one rotator stop 442 may be positioned exterior of the tensioner chamber 422 and/or may be positioned to engage a rotator 410 that rotates the suture tensioner 402 (discussed in more detail below). For example, the rotator 410 can comprise the rotator stop 442 in the form of a component that mates with a corresponding component of the tensioner housing 450 to define the released configuration and/or the fully tensioned configuration. In some examples, using a single rotator stop 442 can enable rotating the suture tensioner 402 through approximately and/or nearly a full rotation before being mechanically restricted by the rotator stop 442. Examples of the rotator 410 are illustrated as the rotator 410c of FIGS. 14A-14B and the rotator 410d of FIGS. 15A-15C and 15E-15H; accordingly, references herein to the rotator 410 may be understood as pertaining to the rotator 410c, the rotator 410d, and/or any other example of a rotator as described herein.

Each post 430 may be configured to engage the release suture 236 in any suitable manner. As illustrated in FIG. 11, each post 430 may be described as comprising a suture engagement surface 432 that engages the release suture 236 during operative use of the suture lock assembly 400 (e.g., while the suture tensioner 402 operates to apply tension to the release suture 236).

In some examples, each suture engagement surface 432 is an outer surface of the respective post 430 and/or is otherwise fixed relative to a remainder of the respective post 430 and/or relative to the connecting body 420. Accordingly, in such examples, the suture tensioner 402 may be configured such that the release suture 236 slides relative to each suture engagement surface 432 as the suture tensioner 402 is rotated relative to the tensioner housing 450. That is, in an example in which each suture engagement surface 432 has a fixed orientation relative to the connecting body 420, rotating the suture tensioner 402 about the tensioner central axis 404 causes each suture engagement surface 432 to revolve about the tensioner central axis 404 as well as to rotate about an axis parallel to the tensioner central axis. Such a motion thus may cause the release suture 236 to slide relative to each suture engagement surface 432 as the posts 430 move to engage the release suture 236.

In other examples, the suture tensioner 402 may be configured such that the release suture 236 remains in at least substantially static contact with each suture engagement surface 432 as the suture tensioner 402 is rotated. As an example, and as illustrated in FIG. 11, the suture tensioner 402 may comprise a pair of rollers 438, each rotatably coupled to a respective post 430 and each comprising a respective suture engagement surface 432. For example, each roller 438 may be a circumferentially surround the respective post 430 and/or may be rotatably coupled to the respective post 430, such as via bearing.

As another example, each post 430 may be rotatably coupled to the connecting body 420. In particular, each post 430 may define a respective suture engagement surface 432, and an entirety (or an at least substantial entirety) of the post 430 may be configured to rotate relative to the connecting body 420.

In such examples, each suture engagement surface 432 may be rotated relative to the connecting body 420 such that the suture engagement surface 432 remains in at least substantially static contact with the release suture 236 as the suture tensioner 402 is rotated. Stated differently, in such examples, the static friction between the release suture 236 and each suture engagement surface 432 may cause each suture engagement surface 432 to rotate relative to the connecting body 420 and/or relative to the respective post 430 as the suture tensioner 402 rotates. Such a configuration thus may facilitate rotating the suture tensioner 402 to increase or decrease the tension in the release suture 236 without the posts 430 tugging on or pushing the release suture 236 due to a sliding friction between the release suture 236 and the suture engagement surfaces 432.

In examples in which each post 430 is circular, the size (e.g., the diameter) of each post 430 may selected based on any of a variety of considerations. For example, larger circular posts 430 may serve to increase a minimum radius of curvature of the release suture 236 within the suture lock assembly 400, which may serve to avoid localized stresses within the release suture 236. Moreover, larger circular posts 430 additionally may correspond to an increased length of the release suture 236 that is wrapped up by the posts 430 as the suture tensioner 402 transitions from the released configuration to the fully tensioned configuration. Alternatively, smaller circular posts 430 may yield a correspondingly wide region between the posts 430 for the release suture 236 to expand into when slackened.

As discussed above, FIG. 13 illustrates an example in which each post 430b has an asymmetric yin-yang shape. FIG. 13 also may be described as representing an example in which each post 430 has a width (e.g., as measured along a direction perpendicular to, and intersecting, the tensioner central axis 404b) that is greater at the leading edge 434b than at the trailing edge 436b.

With reference to the example of FIG. 13, each post 430b may be described as comprising a leading edge 434b and a trailing edge 436b such that each suture engagement surface 432b extends between the leading edge 434b and the trailing edge 436b of the respective post 430b. In particular, compared to the circular posts 430a of FIGS. 12A-12G, the yin-yang shape of the posts 430c of FIG. 13 may yield elongated suture engagement surfaces 432c without significantly diminishing the open space between the posts 430c. Such a configuration may serve to distribute the force of each post 430c upon the release suture 236 across a greater extent of the length of the release suture 236, which may facilitate applying the tension to the release suture 236 with the suture tensioner 402c. Additionally, or alternatively, such a configuration may serve to increase a minimum radius of curvature of the release suture 236 within the suture lock assembly 400c (e.g., relative to the suture lock assembly 400b of FIGS. 12A-12G), which may serve to avoid localized stresses within the release suture 236. Moreover, such a configuration may serve to increase a length of the release suture 236 that is wrapped up by the yin-yang shaped posts 430c per revolution of the suture tensioner 402 relative to a configuration that utilizes circular posts 430.

FIGS. 14A-14B illustrate a third example suture lock assembly 400c, which comprises a suture tensioner 402a in combination with a fourth example quick release suture mechanism 302. In the example of FIGS. 14A-14B, the suture lock assembly 400c is configured such that the tensioner housing 450c extends adjacent to a portion of the delivery apparatus 200a. In particular, FIGS. 14A-14B illustrate an example in which the tensioner housing 450c extends adjacent to, and/or is a component of, a hub assembly 218a of the delivery apparatus 200a that at least partially encloses a sleeve shaft 220a and a pusher shaft 212a. Similar to the hub assembly 218 discussed above, the hub assembly 218a (together with the suture lock assembly 400c) can be used to independently control the pusher shaft 212a and the sleeve shaft 220a while the sleeve handle 224a can control an axial position of the sleeve shaft 220 relative to the pusher shaft 212.

As illustrated in FIGS. 14A-15C and 15E-15H, the suture lock assembly 400 may comprise a rotator 410 configured to be manually actuated by a user to rotate the suture tensioner 402 relative to the tensioner housing 450. In some examples, and as shown in FIGS. 14A and 15E-15H, the rotator 410 is fixedly coupled to each of the posts 430 and/or is integrally formed with the posts 430. Similarly, in some examples, the rotator 410 may be fixedly coupled to the connecting body 420, and/or may comprise and/or define the connecting body 420. In particular, in the example of FIGS. 15E-15H, the rotator 410d, the connecting body 420d, and the posts 430d are integrally formed as a monolithic component such that the rotator 410d extends exterior of the tensioner housing 450d and such the connecting body 420d and the posts 430d extend inside the tensioner housing 450d. As shown in FIG. 15G, the connecting body 420d also can comprise a bottom plate opposite the rotator 410d that restricts the release suture 236 from falling off of the posts 430d. In this manner, the connecting body 420d may be described as operating to retain the release suture 236 within the tensioner housing 450d.

In some examples, one or more components of the suture tensioner 402 are configured to be non-removably coupled to the tensioner housing 450. Stated differently, at least a portion of the suture tensioner 402 may be configured to be coupled to (e.g., assembled with) the tensioner housing 450 in such a manner that the suture tensioner 402 may not be removed from the tensioner housing 450 without damage to the suture tensioner 402 and/or the tensioner housing 450.

As a more specific example, FIG. 14A illustrates an example in which the rotator 410c comprises a plurality of locking tabs 418c that are configured to restrict the rotator 410c from being removed from the tensioner housing 450c.

As another example, FIGS. 15E-15G illustrate an example in which the tensioner housing 450d comprises a plurality of locking tabs 456d that are configured to restrict the rotator 410d from being removed from the tensioner housing 450d.

Accordingly, in such examples, the rotator 410, the connecting body 420, and the posts 430 may be integrally formed (e.g., molded) as a monolithic component that is readily assembled to the tensioner housing 450 by inserting this component into the tensioner housing 450 until the locking tabs (e.g., the locking tabs 418c or the locking tabs 456d) engage and retain the rotator 410 and/or the tensioner housing 450. After the rotator 410, the connecting body 420, and/or the posts 430 are fully inserted into the tensioner housing 450 in this manner, the locking tabs 418 and/or the locking tabs 456 may serve to restrict and/or prevent disassembly of the suture lock assembly 400.

In various examples, the rotator 410 may comprise one or more features to facilitate gripping the rotator 410 and/or rotating the suture tensioner 402 to a desired tensioner configuration. For example, in the example of FIGS. 15A-15C and 15G-15H, the rotator 410d comprises a grip portion 412d configured to facilitate gripping the rotator 410d to manually rotate the suture tensioner 402d relative to the tensioner housing 450d. In the example of FIGS. 15A-15C and 15G-15H, the grip portion 412d comprises a plurality of circumferentially distributed recesses around the circumference of the rotator 410d. In other examples, the grip portion 412 additionally or alternatively may include and/or be any suitable grip feature, examples of which include a textured surface, an indentation, a protrusion, a knob, a lever, a handle, a tab, etc.

In some examples, the suture tensioner 402 and/or the tensioner housing 450 may include one or more features for providing an indication of the rotational position of the rotator 410 relative to the tensioner housing 450. For example, FIG. 14B illustrates an example in which the suture lock assembly 400c comprises an indicator 416c configured to provide a visual indication of the tensioner configuration. In the example of FIG. 14B, each of the rotator 410c and the tensioner housing 450c comprises a portion of the indicator 416c. Specifically, in this example, the indicator 416c includes a mark on the tensioner housing 450c and pair of circumferentially spaced-apart marks on the rotator 410c. Each mark on the rotator 410c thus may be aligned with the mark on the tensioner housing 450c when the suture tensioner 402 is in the released configuration or the fully tensioned configuration. In other examples, the indicator 416 may include and/or be any additional or alternative visual indicator, examples of which include a mark, a printed mark, an embossed mark, a debossed mark, a numerical scale, etc.

Additionally, or alternatively, in some examples, the suture lock assembly 300 and/or the suture lock assembly 400 comprises one or more features to selectively restrict access to the quick release suture mechanism 302. In particular, and as illustrated in dashed lines in FIG. 14B, the suture lock assembly 400c may comprise a safety handle 414c operatively coupled to the rotator 410c and extending away from the rotator 410c. Specifically, when the rotator 410c is in a predetermined rotational orientation relative to the tensioner housing 450c, such as the fully tensioned configuration, the safety handle 414c may cover the quick release suture mechanism 302d to obstruct and/or prevent manual access to the quick release suture mechanism 302d.

In particular, it may be desirable to ensure that the quick release suture mechanism 302 remains in the locked configuration while the suture tensioner 402 is in the fully tensioned configuration. Accordingly, when present, the safety handle 414 (e.g., the safety handle 414c of FIG. 14B) may reduce the likelihood of inadvertently removing the suture anchor cap 320 from the quick release dock port 304 prior to releasing the tension in the release suture 236. In such examples, rotating the rotator 410 to transition the suture tensioner 402 toward and/or to the released configuration (such as by gripping and rotating the safety handle 414) may operate to move the safety handle 414 away from the quick release suture mechanism 302, thereby permitting access to the suture anchor cap 320.

In some examples, the suture lock assembly 400 may include one or more features to restrict and/or prevent unintentional rotation of the rotator 410 relative to the tensioner chamber 422. For example, the suture lock assembly 400 may include a directional selector, such as a mechanism similar to the directional selector 274 of FIGS. 6B-6C, to allow the rotator 410 to rotate in only one direction to prevent rotation in an incorrect direction. In some examples, the directional selector can include a ratchet mechanism, such as a switchable ratchet mechanism that allows the user to select a desired rotational direction. In particular, the directional selector may allow the user to toggle such a switchable ratchet mechanism between a first mode, in which the rotator 410 is rotatable to increase a tension in the release suture 236 and is prevented from rotating to decrease a tension in the release suture 236, and a second mode, in which the rotator 410 is rotatable to decrease a tension in the release suture 236. In some such examples, the rotator is prevented from rotating to increase a tension in the release suture 236 when in the second mode.

As another example (see FIGS. 15F-15G), the suture lock assembly 400d can include a rotator lock mechanism 490d configured to restrict the rotator 410d from rotating away from one or more predetermined configurations (e.g., the fully tensioned configuration). In particular, in this example, the rotator 410d includes a rotator lock channel 424d that is accessible from underneath the rotator 410d, and the rotator receiver 482d includes a rotator lock projection 428d that extends into the rotator lock channel 424d. To illustrate this configuration, FIG. 15F is a cross-sectional view of the suture lock assembly 400 taken through a plane that intersects the rotator 410d at the top of the rotator lock channel 424d. The rotator lock mechanism 490d may be described as including the rotator lock channel 424d and the rotator lock projection 428d.

With reference to FIG. 15F, rotating the rotator 410d relative to the tensioner housing 482d operates to move the rotator lock channel 424d relative to the rotator lock projection 428d, thereby repositioning the rotator lock projection 428d within the rotator lock channel 424d. The rotator 410d additionally can include a detent 426d that serves to increase a radius of the rotator lock channel 424d (e.g., relative to the tensioner central axis 404d) to partially restrict rotation of the rotator 410d relative to the tensioner housing 482d. For example, rotating the rotator 410d to a position at which the rotator lock projection 428d engages the detent 426d may restrict further rotation of the rotator 410d in the same direction unless sufficient torque is applied to the rotator 410d to deflect the rotator lock projection 428d away from the tensioner central axis 404d and/or to deflect the detent 426d toward the tensioner central axis 404d.

Additionally, and as shown in FIG. 15F, the rotator lock channel 424d can include a lock projection receiver 425d in which the rotator lock projection 428d may be received when the rotator 410d is rotated to a position corresponding to the fully tensioned configuration. The lock projection receiver 425 can be partially defined by the detent 426d such that, when the suture tensioner 402d is in the fully tensioned configuration, the rotator 410d is restricted from rotating to release a tension in the release suture unless sufficient torque is applied to the rotator 410d to move the detent 426d past the rotator lock projection 428d.

In the example of FIG. 15F, the rotator lock mechanism 490d includes a single detent 426d that partially defines a single corresponding lock projection receiver 425d, which in turn corresponds to a single predetermined configuration of the suture lock assembly 400d (e.g., the fully tensioned configuration). In other examples, the rotator lock mechanism 490d can operate to define a plurality of such predetermined configurations, such as may correspond to the released configuration or a partially tensioned configuration of the suture lock assembly 400d. For example, such rotator lock mechanisms 490d can include a plurality of detents 426d and/or a plurality of lock projection receivers 425d.

The rotator lock mechanism 490d additionally or alternatively can include any of a variety of other components or mechanisms for defining one or more predetermined configurations of the suture lock assembly 400d. For example, the rotator lock projection 428 may be biased radially inwardly or radially outwardly and may be configured to be received in a groove, a notch, etc. extending radially inwardly or radially outwardly from the rotator lock channel 424d when the rotator 410d is rotated to a position corresponding to a predetermined configuration of the suture lock assembly 400d.

In some examples, and as illustrated in FIGS. 11 and 15D-15G, the suture lock assembly 400 additionally comprises one or more suture centralizers 458 configured to guide the release suture 236 through the tensioner housing 450. For example, when present, each suture centralizer may be configured to engage the tensioner housing 450 (e.g., within the suture entry port 452 or within the suture exit port 454) and to support the release suture 236 through a central bore thereof to maintain at least a portion of the release suture 236 away from an interior surface of the tensioner housing 450.

As further illustrated in FIGS. 11 and 14A-15G, the suture lock assembly 400 additionally may include a flush port 460 fluidly coupled to the suture entry port 452 for supplying a flush fluid into the tensioner housing 450 and/or to a downstream portion of the delivery apparatus 200. Specifically, the flush port 460 may be fluidly coupled to the suture entry port 452 via the tensioner chamber 422. In some such examples, the tensioner housing 450 at least partially defines the flush port 460. In the example of FIGS. 15A-15G, because the quick release suture mechanism 302e enables the release suture 236 to be removed from the implantable device without removing the suture lock assembly 400d from the delivery apparatus connection 306 (shown in FIG. 11), the flush fluid may be delivered to the delivery apparatus 200 via the tensioner housing 450 without a loss of hemostasis.

When present, the flush port 460 may include and/or be any suitable port and/or coupling, such as a port that nominally is sealed and that opens when another component (e.g., a flush fluid source) is operatively coupled to the flush port 460. In some examples, the flush port 460 may include and/or be a Luer lock port and/or a swabbable Luer lock port.

As discussed above, the suture lock assembly 400d of FIGS. 15A-15M represents an example in which the suture lock assembly comprises an example of the quick release suture mechanism 302 (namely, the quick release suture mechanism 302e). In the example of FIGS. 15A-15M, the suture tensioner 402d and the quick release suture mechanism 302e are coupled to one another by a suture lock assembly housing 470d.

As illustrated in FIG. 15D, the suture lock assembly housing 470d comprises a first conduit 484d and a second conduit 486d branching from the first conduit 484d. Specifically, the first conduit may extend fully through the suture lock assembly housing 470d and may be configured to receive a component of a delivery apparatus, such as the sleeve shaft 220 of the delivery apparatus 200. The second conduit 486d leads to the suture tensioner 402d such that the release suture 236 extends to the suture tensioner 402d via the second conduit 486d. In this manner, the first conduit 484d and the second conduit 486d may be analogous to the straight section 242 and the branch 244, respectively, of the adaptor 240 of FIG. 6B.

In the example of FIGS. 15A-15M, the suture lock assembly housing 407d comprises an inner housing 480d and an outer housing 472d that at least substantially encloses the inner housing 480d. More specifically, in this example, the outer housing 472d may comprise an upper outer housing portion 474d and a lower outer housing portion 476d that are configured to be assembled to one another on opposite sides of the inner housing 480d to at least substantially enclose the inner housing 480d.

In the example FIGS. 15A-15M, the inner housing 480d comprises and/or defines each of the first conduit 484d and the second conduit 486d, as well as a rotator receiver 482d that receives at least a portion of the suture tensioner 402d. In particular, and as shown in FIGS. 15E-15G, the rotator receiver 482d defines the locking tabs 456d.

In certain examples, a seal can be included within the suture lock assembly 400, e.g., by using one or more of annular sealing elements (e.g., O-rings) 258 to prevent leakage of blood, saline, or other fluid through the system. For example, as shown in FIGS. 14A and 15E, the suture lock assembly 400c/400d can include an O-ring 258c/258d configured to form a seal between the rotator 410c/410d and the tensioner housing 450c/450d. Additionally, or alternatively, the suture lock assembly 300/400 may include one or more O-rings 258 that are configured to seal the release suture path when the suture lock assembly 300/400 is assembled, allowing for hemostasis when connected to a properly sealed delivery apparatus.

Any of the systems, devices, apparatuses, etc. herein can be sterilized (for example, with heat/thermal, pressure, steam, radiation, and/or chemicals, etc.) to ensure they are safe for use with patients, and any of the methods herein can include sterilization of the associated system, device, apparatus, etc. as one of the steps of the method. Examples heat/thermal sterilization include steam sterilization and autoclaving. Examples of radiation for use in sterilization include, without limitation, gamma radiation, ultra-violet radiation and electron beam. Examples of chemicals for use in sterilization include, without limitation, ethylene oxide, hydrogen peroxide, peracetic acid, formaldehyde, and glutaraldehyde. Sterilization with hydrogen peroxide may be accomplished using hydrogen peroxide plasma, for example.

### Additional Examples of the Disclosed Technology

In view of the above-described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.
Example 1. A quick release suture mechanism comprising a suture anchor cap configured to be selectively coupled to and uncoupled from a quick release dock port; wherein the quick release suture mechanism is configured to be coupled to a suture that is configured to be connected to an implantable device, wherein the quick release suture mechanism is configured to be selectively transitioned between a locked configuration, in which the quick release suture mechanism maintains the suture in a fixed position relative to the quick release dock port, and an unlocked configuration, in which the suture may be removed from the quick release dock port, wherein the quick release suture mechanism is in the locked configuration when the suture anchor cap is operatively coupled to the quick release dock port, and wherein the quick release suture mechanism is in the unlocked configuration when the suture anchor cap is removed from the quick release dock port.
Example 2. The quick release suture mechanism of any example herein, particularly example 1, wherein the quick release suture mechanism is configured to be coupled to a suture lock assembly that comprises a tensioner housing, wherein the quick release suture mechanism comprises the quick release dock port, and wherein the quick release dock port is configured to be attached to a suture exit port of the tensioner housing.
Example 3. The quick release suture mechanism of any example herein, particularly example 2, wherein the quick release suture mechanism is configured such that, during operative use of the quick release suture mechanism, the suture extends from the quick release dock port through the suture exit port and continues through a suture entry port of the suture lock assembly to the implantable device and back to the quick release dock port via the suture entry port and the suture exit port.
Example 4. The quick release suture mechanism of any example herein, particularly any one of examples 1-3, wherein the quick release suture mechanism is configured to be coupled to a suture lock assembly that comprises a tensioner housing and a spool configured to engage the suture, wherein the tensioner housing at least partially receives the spool, and wherein the spool is configured such that, when the quick release suture mechanism is in the locked configuration, rotating the spool relative to the tensioner housing operates to wrap a portion of the suture around the spool to increase a tension in the suture between the suture lock assembly and the implantable device.
Example 5. The quick release suture mechanism of any example herein, particularly any one of examples 1-4, further comprising: the quick release dock port; and a dock port coupling mechanism configured to selectively couple the suture anchor cap to the quick release dock port; wherein one or both of the suture anchor cap and the quick release dock port comprises at least a portion of the dock port coupling mechanism.
Example 6. The quick release suture mechanism of any example herein, particularly example 5, wherein the dock port coupling mechanism comprises a port thread of the quick release dock port and a cap thread of the suture anchor cap, and wherein the cap thread is configured to threadably engage the port thread to selectively couple the suture anchor cap to the quick release dock port.
Example 7. The quick release suture mechanism of any example herein, particularly any one of examples 5-6, wherein the dock port coupling mechanism comprises a bayonet pin and a bayonet slot configured to receive the bayonet pin in a bayonet lock configuration, wherein one of the quick release dock port and the suture anchor cap comprises the bayonet pin, and wherein the other of the quick release dock port and the suture anchor cap comprises the bayonet slot.
Example 8. The quick release suture mechanism of any example herein, particularly example 7, wherein the bayonet pin comprises and terminates at a pin cap that is wider in diameter than a remainder of the bayonet pin, wherein the suture extends between and terminates at each of a suture anchored end and a suture free end, and wherein the quick release suture mechanism is configured such that the suture free end may be wrapped around the bayonet pin such that the pin cap retains the suture on the bayonet pin.
Example 9. The quick release suture mechanism of any example herein, particularly any one of examples 7-8, wherein the bayonet pin comprises a transverse pin bore extending through a diameter of the bayonet pin, and wherein the quick release suture mechanism is configured such that the suture may extend through the transverse pin bore to at least partially retain the suture relative to the bayonet pin.
Example 10. The quick release suture mechanism of any example herein, particularly any one of examples 1-9, wherein the suture anchor cap comprises: an inner plug; an outer skirt circumferentially surrounding the inner plug; and an annular channel defined between the outer skirt and the inner plug, and wherein, when the quick release suture mechanism is in the locked configuration, the annular channel receives at least a portion of the quick release dock port such that the inner plug is received within the quick release dock port and the outer skirt extends circumferentially around the quick release dock port.
Example 11. The quick release suture mechanism of any example herein, particularly any one of examples 1-10, wherein the suture extends between and terminates at each of a suture anchored end and a suture free end, wherein the suture anchor cap comprises a suture anchor location, and wherein the suture anchor cap is configured to be operatively coupled to the suture anchored end at the suture anchor location such that the suture anchored end is restricted from being removed from the suture anchor cap during operative use of the quick release suture mechanism.
Example 12. The quick release suture mechanism of any example herein, particularly example 11, wherein the suture anchored end is configured to be fixedly coupled to the suture anchor location.
Example 13. The quick release suture mechanism of any example herein, particularly any one of examples 11-12, wherein the suture anchored end is configured to be operatively coupled to the suture anchor location via one or more of an adhesive, a cement, a mechanical coupling, a tied coupling, and a mechanical obstruction.
Example 14. The quick release suture mechanism of any example herein, particularly any one of examples 11-13, wherein the quick release suture mechanism is configured such that: when the quick release suture mechanism is in the locked configuration, the suture free end is at least substantially fixed in position relative to the quick release dock port, and when the quick release suture mechanism is in the unlocked configuration, the suture free end is free to move through the quick release dock port.
Example 15. The quick release suture mechanism of any example herein, particularly any one of examples 11-14, further comprising a dock port coupling mechanism configured to selectively couple the suture anchor cap to the quick release dock port, wherein the dock port coupling mechanism comprises a port thread of the quick release dock port and a cap thread of the suture anchor cap, wherein the cap thread is configured to threadably engage the port thread to selectively couple the suture anchor cap to the quick release dock port, and wherein the quick release suture mechanism is configured such that, when the quick release suture mechanism is in the locked configuration, the suture free end extends between, and is locked in position by, the port thread and the cap thread.
Example 16. The quick release suture mechanism of any example herein, particularly any one of examples 11-15, wherein the suture anchor cap defines an inner bore, and wherein one or both of the suture anchored end and the suture free end extends at least partially through the inner bore.
Example 17. The quick release suture mechanism of any example herein, particularly example 16, wherein the inner bore comprises the suture anchor location.
Example 18. The quick release suture mechanism of any example herein, particularly any one of examples 16-17, wherein the suture anchor cap comprises an inner plug that defines at least a portion of the inner bore.
Example 19. The quick release suture mechanism of any example herein, particularly any one of examples 16-18, wherein the inner bore is a first inner bore, wherein the suture anchor cap further comprises a second inner bore, wherein the suture anchored end extends at least partially through the first inner bore, and wherein the suture free end extends at least partially through the second inner bore when the quick release suture mechanism is in the locked configuration.
Example 20. The quick release suture mechanism of any example herein, particularly any one of examples 11-19, wherein the inner bore extends fully through a length of the suture anchor cap.
Example 21. The quick release suture mechanism of any example herein, particularly any one of examples 11-19, wherein the inner bore extends through only a portion of a length of the suture anchor cap.
Example 22. The quick release suture mechanism of any example herein, particularly any one of examples 11-21, wherein the quick release suture mechanism is configured such that the suture free end extends at least substantially exterior of the inner bore when the quick release suture mechanism is in the locked configuration.
Example 23. The quick release suture mechanism of any example herein, particularly any one of examples 11-22, wherein the quick release dock port is configured to be attached to a suture exit port of a tensioner housing, wherein the suture anchor cap comprises: an inner plug; an outer skirt circumferentially surrounding the inner plug; and an annular channel defined between the outer skirt and the inner plug; wherein, when the quick release suture mechanism is in the locked configuration, the annular channel receives at least a portion of the quick release dock port such that the inner plug is received within the quick release dock port and the outer skirt extends circumferentially around the quick release dock port, and wherein the quick release suture mechanism is configured such that, when the quick release suture mechanism is in the locked configuration, the suture free end extends from the suture exit port through the inner bore, around the outer skirt, into the annular channel, and again through the inner bore.
Example 24. The quick release suture mechanism of any example herein, particularly any one of examples 1-23, wherein the suture anchor cap comprises a grip feature configured to facilitate gripping the suture anchor cap to transition the quick release suture mechanism between the locked configuration and the unlocked configuration.
Example 25. The quick release suture mechanism of any example herein, particularly example 24, wherein the grip feature comprises one or more of a textured surface, an indentation, a protrusion, a knob, a lever, a handle, and a tab.
Example 26. The quick release suture mechanism of any example herein, particularly any one of examples 1-25, further comprising a gasket that is configured to form an at least substantially fluid-tight seal to restrict leakage out of the quick release dock port.
Example 27. The quick release suture mechanism of any example herein, particularly example 26, wherein the quick release suture mechanism is configured to be coupled to a suture lock assembly that comprises a tensioner housing, and wherein the gasket is configured to form an at least substantially fluid-tight seal between the suture anchor cap and the tensioner housing when the quick release suture mechanism is in the locked configuration.
Example 28. The quick release suture mechanism of any example herein, particularly any one of examples 26-27, wherein the gasket is configured to form an at least substantially fluid-tight seal to restrict fluid from exiting the quick release dock port when the quick release suture mechanism is in the unlocked configuration.
Example 29. The quick release suture mechanism of any example herein, particularly any one of examples 26-28, wherein the quick release dock port is configured to be attached to a suture exit port of a tensioner housing, and wherein the gasket is received within one or both of the suture exit port and the quick release dock port.
Example 30. The quick release suture mechanism of any example herein, particularly any one of examples 26-29, wherein the quick release dock port is configured to be attached to a suture exit port of a tensioner housing, and wherein the gasket is a suture centralizer that operates to maintain the suture in a central region of the suture exit port to align the suture with an inner bore of the suture anchor cap.
Example 31. The quick release suture mechanism of any example herein, particularly any one of examples 26-30, further comprising a dock port coupling mechanism configured to selectively couple the suture anchor cap to the quick release dock port, wherein the dock port coupling mechanism comprises a bayonet pin and a bayonet slot configured to receive the bayonet pin in a bayonet lock configuration, wherein one of the quick release dock port and the suture anchor cap comprises the bayonet pin, wherein the other of the quick release dock port and the suture anchor cap comprises the bayonet slot, and wherein, when the quick release suture mechanism is in the locked configuration, the gasket urges the suture anchor cap radially outward to urge the bayonet pin into engagement with the bayonet slot.
Example 32. A suture lock assembly, comprising: a spool configured to engage a suture that is configured to be connected to an implantable device; and a quick release suture mechanism; wherein the quick release suture mechanism is the quick release suture mechanism of any one of examples 1-31.
Example 33. The suture lock assembly of any example herein, particularly example 32, further comprising a tensioner housing that at least partially receives the spool, wherein the tensioner housing comprises a suture entry port and a suture exit port, and wherein the quick release suture mechanism is attached to the suture exit port.
Example 34. The suture lock assembly of any example herein, particularly example 33, wherein the tensioner housing comprises the quick release dock port.
Example 35. The suture lock assembly of any example herein, particularly any one of examples 33-34, wherein the quick release dock port and the suture exit port are integrally formed.
Example 36. The suture lock assembly of any example herein, particularly any one of examples 33-35, wherein the suture lock assembly is configured such that the suture extends between the implantable device and the spool via the suture entry port.
Example 37. The suture lock assembly of any example herein, particularly any one of examples 33-36, wherein the suture lock assembly is configured such that the suture extends between the spool and the quick release suture mechanism via the suture exit port.
Example 38. The suture lock assembly of any example herein, particularly any one of examples 32-37, wherein the suture lock assembly is configured to be operatively coupled to a delivery apparatus configured to deliver the implantable device to a target implantation site within a patient's body, and wherein the tensioner housing is configured to be operatively coupled to a delivery apparatus connection of the delivery apparatus such that the suture extends between the spool and the implantable device via the delivery apparatus connection.
Example 39. The suture lock assembly of any example herein, particularly example 38, further comprising a tensioner housing that at least partially receives the spool, wherein the tensioner housing comprises a suture entry port and a suture exit port, wherein the quick release suture mechanism is attached to the suture exit port, and wherein the suture entry port is configured to be operatively coupled to the delivery apparatus connection.
Example 40. The suture lock assembly of any example herein, particularly any one of examples 33-39, further comprising one or more suture centralizers configured to guide the suture through the tensioner housing.
Example 41. The suture lock assembly of any example herein, particularly example 40, wherein at least one suture centralizer is configured to maintain at least a portion of the suture away from an interior surface of the tensioner housing.
Example 42. The suture lock assembly of any example herein, particularly any one of examples 40-41, wherein at least one suture centralizer engages the tensioner housing within the suture entry port.
Example 43. The suture lock assembly of any example herein, particularly any one of examples 40-42, wherein at least one suture centralizer engages the tensioner housing within the suture exit port.
Example 44. The suture lock assembly of any example herein, particularly any one of examples 33-43, wherein the spool is configured to be selectively rotated relative to the tensioner housing to selectively increase a length of the suture that is enclosed by the tensioner housing.
Example 45. The suture lock assembly of any example herein, particularly any one of examples 33-44, further comprising a rotator that is configured to be manually actuated by a user to rotate the spool relative to the tensioner housing.
Example 46. The suture lock assembly of any example herein, particularly example 45, wherein the rotator is fixedly coupled to the spool.
Example 47. The suture lock assembly of any example herein, particularly any one of examples 45-46, wherein at least a portion of the rotator extends exterior of the tensioner housing.
Example 48. The suture lock assembly of any example herein, particularly any one of examples 45-47, wherein the rotator comprises a grip portion configured to facilitate gripping the rotator to manually rotate the spool relative to the tensioner housing.
Example 49. The suture lock assembly of any example herein, particularly example 48, wherein the grip portion comprises one or more of a textured surface, an indentation, a protrusion, a knob, a lever, a handle, and a tab.
Example 50. The suture lock assembly of any example herein, particularly any one of examples 45-49, further comprising a safety handle that is operatively coupled to the rotator and that extends away from the rotator, wherein the safety handle covers the quick release suture mechanism to obstruct access to the quick release suture mechanism when the rotator is a predetermined rotational orientation relative to the tensioner housing.
Example 51. The suture lock assembly of any example herein, particularly example 50, wherein the predetermined rotational orientation corresponds to a configuration in which the spool operates to apply tension to the suture.
Example 52. The suture lock assembly of any example herein, particularly any one of examples 50-51, wherein the suture lock assembly is configured such that rotating the rotator away from the predetermined rotational orientation moves the safety handle away from the quick release suture mechanism to permit access to the suture anchor cap.
Example 53. The suture lock assembly of any example herein, particularly any one of examples 45-52, further comprising a directional selector configured to allow the rotator to rotate in only one direction to prevent rotation in an incorrect direction.
Example 54. The suture lock assembly of any example herein, particularly example 53, wherein the directional selector comprises a ratchet mechanism.
Example 55. The suture lock assembly of any example herein, particular any one of examples 53-54, wherein the directional selector is configured to be toggled between a first mode, in which the rotator is rotatable to increase a tension in the suture and is prevented from rotating to decrease a tension in the suture, and a second mode, in which the rotator is rotatable to decrease a tension in the suture and is prevented from rotating to increase a tension in the suture.
Example 56. The suture lock assembly of any example herein, particularly any one of examples 33-55, further comprising a flush port fluidly coupled to the suture entry port.
Example 57. The suture lock assembly of any example herein, particularly example 56, wherein the tensioner housing at least partially defines the flush port.
Example 58. A delivery apparatus for a prosthetic implant, comprising the quick release suture mechanism of any one of examples 1-31 or the suture lock assembly of any one of examples 32-57.
Example 59. A suture lock assembly, comprising: a suture tensioner configured to engage a suture that is configured to be connected to an implantable device, wherein the suture tensioner is configured to be selectively rotated about a tensioner central axis to collect a length of the suture; and wherein the suture tensioner comprises: a pair of spaced-apart posts, each post configured to revolve around the tensioner central axis as the suture tensioner rotates; and a connecting body coupled to each of the posts.
Example 60. The suture lock assembly of any example herein, particularly example 59, further comprising a tensioner housing that at least partially receives the suture tensioner, wherein the suture tensioner is configured to rotate relative to the tensioner housing, and wherein the tensioner housing comprises a suture entry port such that the suture extends between the implantable device and the suture tensioner via the suture entry port.
Example 61. The suture lock assembly of any example herein, particularly example 60, wherein the suture lock assembly is configured such that, during operative use of the suture lock assembly, the suture extends from the suture tensioner through the suture entry port to the implantable device and back to the suture tensioner via the suture entry port.
Example 62. The suture lock assembly of any example herein, particularly any one of examples 59-61, wherein the suture tensioner is configured such that rotating the suture tensioner operates to wrap a portion of the suture around the pair of posts to increase a tension in the suture between the suture tensioner and the implantable device.
Example 63. The suture lock assembly of any example herein, particularly any one of examples 59-62, wherein the suture lock assembly is configured such that at least a portion of the suture is fixedly coupled to one of the posts.
Example 64. The suture lock assembly of any example herein, particularly any one of examples 59-63, wherein the suture extends between and terminates at each of a first end and a second end, and wherein one or both of the first end and the second end is fixedly coupled to a suture anchor location of the suture tensioner.
Example 65. The suture lock assembly of any example herein, particularly example 64, wherein at least one of the posts comprises the suture anchor location.
Example 66. The suture lock assembly of any example herein, particularly any one of examples 59-65, wherein the suture lock assembly is configured such that the suture tensioner may be rotated to a position in which the posts are spaced apart from the suture.
Example 67. The suture lock assembly of any example herein, particularly any one of examples 60-66, wherein the tensioner housing further comprises a suture exit port such that the suture extends through the tensioner housing between the suture entry port and the suture exit port.
Example 68. The suture lock assembly of any example herein, particularly any one of examples 59-67, wherein the suture lock assembly comprises a rotator that is configured to be manually actuated by a user to rotate the suture tensioner.
Example 69. The suture lock assembly of any example herein, particularly example 68, wherein the rotator is fixedly coupled to each of the posts.
Example 70. The suture lock assembly of any example herein, particularly any one of examples 68-69, wherein the rotator and the posts are integrally formed.
Example 71. The suture lock assembly of any example herein, particularly any one of examples 68-70, wherein the rotator is fixedly coupled to the connecting body.
Example 72. The suture lock assembly of any example herein, particularly any one of examples 68-71, wherein the rotator comprises the connecting body.
Example 73. The suture lock assembly of any example herein, particularly any one of examples 68-72, wherein the rotator comprises a grip portion configured to facilitate gripping the rotator to manually rotate the suture tensioner.
Example 74. The suture lock assembly of any example herein, particularly example 73, wherein the grip portion comprises one or more of a textured surface, an indentation, a protrusion, a knob, a lever, a handle, and a tab.
Example 75. The suture lock assembly of any example herein, particularly any one of examples 68-74, further comprising a directional selector configured to allow the rotator to rotate in only one direction to prevent rotation in an incorrect direction.
Example 76. The suture lock assembly of any example herein, particularly example 75, wherein the directional selector comprises a ratchet mechanism.
Example 77. The suture lock assembly of any example herein, particular any one of examples 75-76, wherein the directional selector is configured to be toggled between a first mode, in which the rotator is rotatable to increase a tension in the suture and is prevented from rotating to decrease a tension in the suture, and a second mode, in which the rotator is rotatable to decrease a tension in the suture and is prevented from rotating to increase a tension in the suture.
Example 78. The suture lock assembly of any example herein, particularly any one of examples 68-77, further comprising a tensioner housing that at least partially receives the suture tensioner, wherein the suture tensioner is configured to rotate relative to the tensioner housing, and wherein at least a portion of the rotator extends exterior of the tensioner housing.
Example 79. The suture lock assembly of any example herein, particularly any one of examples 68-78, further comprising a tensioner housing that at least partially receives the suture tensioner, wherein the suture tensioner is configured to rotate relative to the tensioner housing, and wherein one or both of the tensioner housing and the rotator comprises one or more locking tabs configured to restrict the rotator from being removed from the tensioner housing.
Example 80. The suture lock assembly of any example herein, particularly any one of examples 59-79, wherein the suture tensioner is configured to rotate to transition the suture tensioner among a plurality of tensioner configurations defined between and comprising a released configuration, in which the posts are positioned to exert no force on the suture, and a fully tensioned configuration, in which the posts are positioned to engage the suture to yield a maximum tension in the suture when the suture is connected to the implantable device.
Example 81. The suture lock assembly of any example herein, particularly example 80, wherein the suture tensioner is configured to rotate through an angle that is one or more of less than 180 degrees, about 180 degrees, more than 180 degrees, less than 360 degrees, about 360 degrees, and more than 360 degrees to transition the suture tensioner between the released configuration and the fully tensioned configuration.
Example 82. The suture lock assembly of any example herein, particularly any one of examples 80-81, further comprising one or more rotator stops that are configured to engage a portion of suture lock assembly to mechanically restrict rotation of the suture tensioner to define one or both of the released configuration and the fully tensioned configuration.
Example 83. The suture lock assembly of any example herein, particularly example 82, further comprising a tensioner housing that at least partially receives the suture tensioner, wherein the suture tensioner is configured to rotate relative to the tensioner housing, and wherein the one or more rotator stops are fixed in position relative to the tensioner housing.
Example 84. The suture lock assembly of any example herein, particularly example 83, further comprising a rotator that is configured to be manually actuated by a user to rotate the suture tensioner relative to the tensioner housing, and wherein at least one rotator stop of the one or more rotator stops is configured to engage the rotator to define one or both of the released configuration and the fully tensioned configuration.
Example 85. The suture lock assembly of any example herein, particularly any one of examples 82-84, wherein at least one rotator stop of the one or more rotator stops is configured to engage at least one post of the pair of spaced-apart posts to define one or both of the released configuration and the fully tensioned configuration.
Example 86. The suture lock assembly of any example herein, particularly any one of examples 80-85, further comprising an indicator configured to provide a visual indication of the tensioner configuration.
Example 87. The suture lock assembly of any example herein, particularly example 86, further comprising: a tensioner housing that at least partially receives the suture tensioner; and a rotator that is configured to be manually actuated by a user to rotate the suture tensioner relative to the tensioner housing, and wherein one or both of the rotator and the tensioner housing comprises at least a portion of the indicator.
Example 88. The suture lock assembly of any example herein, particularly any one of examples 86-87, wherein the indicator is configured to provide an indication that the suture tensioner is in one or both of the released configuration and the fully tensioned configuration.
Example 89. The suture lock assembly of any example herein, particularly any one of examples 86-88, wherein the indicator comprises one or more of a mark, a printed mark, an embossed mark, a debossed mark, a numerical scale, and a pair of visual indicators that are aligned when the suture tensioner is in one or both of the released configuration and the fully tensioned configuration.
Example 90. The suture lock assembly of any example herein, particularly any one of examples 80-89, further comprising a tensioner housing that at least partially receives the suture tensioner, wherein the suture tensioner is configured to rotate relative to the tensioner housing, wherein the tensioner housing comprises a suture entry port such that the suture extends between the implantable device and the suture tensioner via the suture entry port, wherein the tensioner housing further comprises a suture exit port such that the suture extends through the tensioner housing between the suture entry port and the suture exit port, and wherein, when the suture tensioner is in the released configuration, one or both of the posts are spaced apart from a straight line connecting the suture entry port and the suture exit port.
Example 91. The suture lock assembly of any example herein, particularly any one of examples 80-90, further comprising a tensioner housing that at least partially receives the suture tensioner, wherein the suture tensioner is configured to rotate relative to the tensioner housing, wherein the tensioner housing comprises a suture entry port such that the suture extends between the implantable device and the suture tensioner via the suture entry port, wherein the tensioner housing further comprises a suture exit port such that the suture extends through the tensioner housing between the suture entry port and the suture exit port, and wherein, when the suture tensioner is in the released configuration, one or both of the posts are tangent to a straight line connecting the suture entry port and the suture exit port.
Example 92. The suture lock assembly of any example herein, particularly any one of examples 80-91, further comprising a tensioner housing that at least partially receives the suture tensioner, wherein the suture tensioner is configured to rotate relative to the tensioner housing, wherein the tensioner housing comprises a tensioner chamber that encloses at least a portion of the posts and a portion of the suture, and wherein, when the suture tensioner is in the released configuration, the suture is free to occupy a region of the tensioner chamber between the posts without obstruction by a portion of the suture tensioner.
Example 93. The suture lock assembly of any example herein, particularly any one of examples 59-92, further comprising: a rotator that is configured to be manually actuated by a user to rotate the suture tensioner; and a rotator lock mechanism configured to restrict the rotator from rotating away from one or more predetermined configurations.
Example 94. The suture lock assembly of any example herein, particularly example 93, wherein the suture tensioner is configured to rotate to transition the suture tensioner among a plurality of tensioner configurations defined between and comprising a released configuration, in which the posts are positioned to exert no force on the suture, and a fully tensioned configuration, in which the posts are positioned to engage the suture to yield a maximum tension in the suture when the suture is connected to the implantable device, and wherein the rotator lock mechanism is configured to restrict the rotator from rotating away from the fully tensioned configuration.
Example 95. The suture lock assembly of any example herein, particularly any one of examples 93-94, further comprising a tensioner housing that at least partially receives the suture tensioner, wherein the rotator comprises a rotator lock channel, wherein the tensioner housing comprises a rotator lock projection that extends into the rotator lock channel, and wherein the rotator lock mechanism comprises the rotator lock channel and the rotator lock projection.
Example 96. The suture lock assembly of any example herein, particularly example 95, wherein rotating the rotator relative to the tensioner housing operates to move the rotator lock channel relative to the rotator lock projection.
Example 97. The suture lock assembly of any example herein, particularly any one of examples 95-96, wherein the rotator comprises a detent that increases a radius of the rotator lock channel to partially restrict rotation of the rotator relative to the tensioner housing.
Example 98. The suture lock assembly of any example herein, particularly example 97, wherein the rotator lock channel comprises a lock projection receiver in which the rotator lock projection is received when the rotator is rotated to a position corresponding to a fully tensioned configuration, and wherein the detent partially defines the lock projection receiver.
Example 99. The suture lock assembly of any example herein, particularly any one of examples 59-98, wherein each post has a shape that is one or more of cylindrical, conical, and frusto-conical.
Example 100. The suture lock assembly of any example herein, particularly any one of examples 59-99, wherein each post has a cross-sectional shape that is one or more of curved, circular, oval, elliptical, and yin-yang shaped.
Example 101. The suture lock assembly of any example herein, particularly any one of examples 59-100, wherein the pair of posts are at least substantially identical to one another.
Example 102. The suture lock assembly of any example herein, particularly any one of examples 59-101, wherein each post comprises a suture engagement surface that engages the suture during operative use of the suture lock assembly.
Example 103. The suture lock assembly of any example herein, particularly example 102, wherein the suture tensioner is configured such that the suture slides relative to each suture engagement surface as the suture tensioner is rotated.
Example 104. The suture lock assembly of any example herein, particularly any one of examples 102-103, wherein the suture tensioner is configured such that the suture remains in static contact with each suture engagement surface as the suture tensioner is rotated.
Example 105. The suture lock assembly of any example herein, particularly any one of examples 102-104, wherein the suture tensioner comprises a pair of rollers, each roller rotatably coupled to a respective post, wherein each roller comprises a respective suture engagement surface, and wherein each roller is configured to rotate relative to the respective post to maintain the suture in static contact with the respective suture engagement surface as the suture tensioner is rotated.
Example 106. The suture lock assembly of any example herein, particularly any one of examples 102-105, wherein each post comprises a leading edge and a trailing edge, and wherein the suture engagement surface extends between the leading edge and the trailing edge.
Example 107. The suture lock assembly of any example herein, particularly example 106, wherein each post has a width, as measured along a direction that is perpendicular to the tensioner central axis and that intersects the tensioner central axis, that is greater at the leading edge of the post than at the trailing edge of the post.
Example 108. The suture lock assembly of any example herein, particularly any one of examples 59-107, wherein each post is rotatably coupled to the connecting body.
Example 109. The suture lock assembly of any example herein, particularly any one of examples 60-108, wherein the suture lock assembly is configured to be operatively coupled to a delivery apparatus configured to deliver the implantable device to a target implantation site within a patient's body, and wherein the tensioner housing is configured to be operatively coupled to a delivery apparatus connection of the delivery apparatus such that the suture extends between the suture tensioner and the implantable device via the delivery apparatus connection.
Example 110. The suture lock assembly of any example herein, particularly example 109, wherein the suture entry port is configured to be operatively coupled to the delivery apparatus connection.
Example 111. The suture lock assembly of any example herein, particularly any one of examples 60-110, further comprising one or more suture centralizers configured to guide the suture through the tensioner housing.
Example 112. The suture lock assembly of any example herein, particularly example 111, wherein at least one suture centralizer is configured to maintain at least a portion of the suture away from an interior surface of the tensioner housing.
Example 113. The suture lock assembly of any example herein, particularly any one of examples 111-112, wherein at least one suture centralizer engages the tensioner housing within the suture entry port.
Example 114. The suture lock assembly of any example herein, particularly any one of examples 111-113, wherein the tensioner housing further comprises a suture exit port such that the suture extends through the tensioner housing between the suture entry port and the suture exit port, and wherein at least one suture centralizer engages the tensioner housing within the suture exit port.
Example 115. The suture lock assembly of any example herein, particularly any one of examples 60-114, further comprising a flush port fluidly coupled to the suture entry port.
Example 116. The suture lock assembly of any example herein, particularly example 115, wherein the tensioner housing at least partially defines the flush port.
Example 117. The suture lock assembly of any example herein, particularly any one of examples 115-116, wherein the tensioner housing comprises a tensioner chamber that encloses a portion of the suture, and wherein the flush port is fluidly coupled to the suture entry port via the tensioner chamber.
Example 118. The suture lock assembly of any example herein, particularly any one of examples 60-117, wherein the tensioner housing further comprises a suture exit port such that the suture extends through the tensioner housing between the suture entry port and the suture exit port, wherein the suture lock assembly further comprises a quick release suture mechanism coupled to the suture exit port, and wherein the quick release suture mechanism is the quick release suture mechanism of any of examples 1-31.
Example 119. The suture lock assembly of any example herein, particularly example 118, further comprising a suture lock assembly housing that operatively couples the suture tensioner and the quick release suture mechanism to one another.
Example 120. The suture lock assembly of any example herein, particularly example 119, wherein the suture lock assembly housing comprises: a first conduit configured to receive a sleeve shaft of a delivery apparatus; and a second conduit branching from the first conduit; and wherein the suture extends to the suture tensioner via the second conduit.
Example 121. The suture lock assembly of any example herein, particularly any one of examples 119-120, wherein the suture lock assembly housing comprises an inner housing and an outer housing that at least substantially encloses the inner housing.
Example 122. The suture lock assembly of any example herein, particularly example 121, wherein the inner housing comprises: a first conduit configured to receive a sleeve shaft of a delivery apparatus; and a second conduit branching from the first conduit; and wherein the suture extends to the suture tensioner via the second conduit.
Example 123. The suture lock assembly of any example herein, particularly any one of examples 121-122, wherein the outer housing comprises an upper outer housing portion and a lower outer housing portion that are configured to be assembled to one another on opposite sides of the inner housing to at least substantially enclose the inner housing.
Example 124. The suture lock assembly of any example herein, particularly any one of examples 121-123, wherein the inner housing comprises a rotator receiver that receives at least a portion of the suture tensioner.
Example 125. The suture lock assembly of any example herein, particularly any one of examples 121-124, wherein the suture lock assembly comprises a rotator that is configured to be manually actuated by a user to rotate the suture tensioner, and wherein the inner housing comprises one or more locking tabs configured to restrict the rotator from being removed from the rotator receiver.
Example 126. The quick release suture mechanism of any example herein, particularly any one of examples 1-31, wherein the quick release suture mechanism is sterilized.
Example 127. A method comprising: sterilizing the quick release suture mechanism of any one of examples 1-31.
Example 128. The suture lock assembly of any example herein, particularly any one of examples 32-57 and examples 69-125, wherein the suture lock assembly is sterilized.
Example 129. A method comprising: sterilizing the suture lock assembly of any one of examples 32-57 and examples 59-125.
Example 130. The delivery apparatus of any example herein, particularly example 58, wherein the delivery apparatus is sterilized.
Example 131. A method comprising: sterilizing the delivery apparatus of example 130.

The features described herein with regard to any example can be combined with other features described in any one or more of the other examples, unless otherwise stated. For example, any one or more of the features of one quick release suture mechanism can be combined with any one or more features of another quick release suture mechanism. As another example, any one or more features of one suture lock assembly can be combined with any one or more features of another suture lock assembly and/or with any one or more features of a quick release suture mechanism. Additionally, any one or more features of one delivery apparatus can be combined with any one or more features of another delivery apparatus, with one or more features of a suture lock assembly, and/or with one or more features of a quick release suture mechanism.

In view of the many possible ways in which the principles of the disclosure may be applied, it should be recognized that the illustrated configurations depict examples of the disclosed technology and should not be taken as limiting the scope of the disclosure nor the claims. Rather, the scope of the claimed subject matter is defined by the following claims and their equivalents.

1. A suture lock assembly, comprising:
a suture tensioner configured to engage a suture that is configured to be connected to an implantable device,
wherein the suture tensioner is configured to be selectively rotated about a tensioner central axis to collect a length of the suture, and wherein the suture tensioner comprises:
a pair of spaced-apart posts, each post configured to revolve around the tensioner central axis as the suture tensioner rotates; and
a connecting body coupled to each of the posts.

2. The suture lock assembly of claim 1, wherein the suture tensioner is configured such that rotating the suture tensioner operates to wrap a portion of the suture around the pair of posts to increase a tension in the suture between the suture tensioner and the implantable device.

3. The suture lock assembly of claim 1, wherein the suture tensioner is configured to rotate to transition the suture tensioner among a plurality of tensioner configurations defined between and comprising a released configuration, in which the posts are positioned to exert no force on the suture, and a fully tensioned configuration, in which the posts are positioned to engage the suture to yield a maximum tension in the suture when the suture is connected to the implantable device.

4. The suture lock assembly of claim 3, wherein the suture tensioner is configured to rotate through an angle that is about 180 degrees to transition the suture tensioner between the released configuration and the fully tensioned configuration.

5. The suture lock assembly of claim 3, further comprising one or more rotator stops that are configured to engage at least one of the posts to define one or both of the released configuration and the fully tensioned configuration.

6. The suture lock assembly of claim 5, further comprising a tensioner housing that at least partially receives the suture tensioner, wherein the suture tensioner is configured to rotate relative to the tensioner housing, and wherein the one or more rotator stops are fixed in position relative to the tensioner housing.

7. The suture lock assembly of claim 3, further comprising an indicator configured to provide a visual indication of the tensioner configuration.

8. The suture lock assembly of claim 3, further comprising a tensioner housing that at least partially receives the suture tensioner, wherein the suture tensioner is configured to rotate relative to the tensioner housing, wherein the tensioner housing comprises:
a suture entry port such that the suture extends between the implantable device and the suture tensioner via the suture entry port; and
a suture exit port such that the suture extends through the tensioner housing between the suture entry port and the suture exit port, and
wherein, when the suture tensioner is in the released configuration, one or both of the posts are one of:
(i) tangent to a straight line connecting the suture entry port and the suture exit port; or
(ii) spaced apart from the straight line connecting the suture entry port and the suture exit port.

9. The suture lock assembly of claim 3, further comprising a tensioner housing that at least partially receives the suture tensioner, wherein the suture tensioner is configured to rotate relative to the tensioner housing, wherein the tensioner housing comprises a tensioner chamber that encloses at least a portion of the posts and a portion of the suture, and wherein, when the suture tensioner is in the released configuration, the suture is free to occupy a region of the tensioner chamber between the posts without obstruction by a portion of the suture tensioner.

10. The suture lock assembly of claim 1, further comprising a tensioner housing that at least partially receives the suture tensioner, wherein the suture tensioner is configured to rotate relative to the tensioner housing, and wherein the tensioner housing comprises a suture entry port such that the suture extends between the implantable device and the suture tensioner via the suture entry port.

11. The suture lock assembly of claim 10, wherein the suture lock assembly is configured to be operatively coupled to a delivery apparatus configured to deliver the implantable device to a target implantation site within a patient's body, and wherein the suture entry port is configured to be operatively coupled to a delivery apparatus connection of the delivery apparatus such that the suture extends between the suture tensioner and the implantable device via the delivery apparatus connection.

12. The suture lock assembly of any one of claim 10, further comprising a flush port fluidly coupled to the suture entry port, wherein the tensioner housing comprises a tensioner chamber that encloses a portion of the suture, and wherein the flush port is fluidly coupled to the suture entry port via the tensioner chamber.

13. A suture lock assembly, comprising:
a suture tensioner configured to engage a suture that is configured to be connected to an implantable device;
a tensioner housing that at least partially receives the suture tensioner, wherein the tensioner housing comprises a suture entry port and a suture exit port such that the suture extends through the tensioner housing between the suture entry port and the suture exit port; and
a quick release suture mechanism coupled to the suture exit port;
wherein the suture tensioner is configured to be selectively rotated relative to the tensioner housing about a tensioner central axis to selectively increase a length of the suture that is enclosed by the tensioner housing,
wherein the suture tensioner comprises:
a pair of spaced-apart posts, each post configured to revolve around the tensioner central axis as the suture tensioner rotates relative to the tensioner housing; and
a connecting body coupled to each of the posts;
wherein the quick release suture mechanism comprises:
a quick release dock port configured to be attached the suture exit port; and
a suture anchor cap configured to be selectively coupled to and uncoupled from the quick release dock port;
wherein the quick release suture mechanism is configured to be selectively transitioned between a locked configuration, in which the quick release suture mechanism maintains the suture in a fixed position relative to the suture exit port, and an unlocked configuration, in which the suture may be removed from the suture exit port,
wherein the quick release suture mechanism is in the locked configuration when the suture anchor cap is operatively coupled to the quick release dock port,
wherein the quick release suture mechanism is in the unlocked configuration when the suture anchor cap is removed from the quick release dock port, wherein the suture extends between and terminates at each of a suture anchored end and a suture free end,
wherein the suture anchor cap comprises a suture anchor location, and
wherein the suture anchor cap is configured to be operatively coupled to the suture anchored end at the suture anchor location such that the suture anchored end is restricted from being removed from the suture anchor cap during operative use of the quick release suture mechanism.

14. The suture lock assembly of claim 13, further comprising a dock port coupling mechanism configured to selectively couple the suture anchor cap to the quick release dock port, wherein the dock port coupling mechanism comprises a port thread of the quick release dock port and a cap thread of the suture anchor cap, wherein the cap thread is configured to threadably engage the port thread to selectively couple the suture anchor cap to the quick release dock port, and wherein the quick release suture mechanism is configured such that, when the quick release suture mechanism is in the locked configuration, the suture free end extends between, and is locked in position by, the port thread and the cap thread.

15. The suture lock assembly of claim 13, further comprising a dock port coupling mechanism configured to selectively couple the suture anchor cap to the quick release dock port, wherein the dock port coupling mechanism comprises a bayonet pin and a bayonet slot configured to receive the bayonet pin in a bayonet lock configuration, wherein one of the quick release dock port and the suture anchor cap comprises the bayonet pin, and wherein the other of the quick release dock port and the suture anchor cap comprises the bayonet slot.

16. The suture lock assembly of claim 13, wherein the suture anchor cap defines an inner bore, and wherein one or both of the suture anchored end and the suture free end extends at least partially through the inner bore.

17. The suture lock assembly of claim 16, wherein the suture anchor cap comprises:
an inner plug;
an outer skirt circumferentially surrounding the inner plug; and
an annular channel defined between the outer skirt and the inner plug,
wherein, when the quick release suture mechanism is in the locked configuration, the annular channel receives at least a portion of the quick release dock port such that the inner plug is received within the quick release dock port and the outer skirt extends circumferentially around the quick release dock port, and wherein the inner plug defines at least a portion of the inner bore.

18. The suture lock assembly of claim 16, wherein the inner bore is a first inner bore, wherein the suture anchor cap further comprises a second inner bore, wherein the suture anchored end extends at least partially through the first inner bore, and wherein the suture free end extends at least partially through the second inner bore when the quick release suture mechanism is in the locked configuration.

19. The suture lock assembly of claim 13, further comprising a rotator that is configured to be manually actuated by a user to rotate the suture tensioner relative to the tensioner housing, wherein the rotator is configured to rotate the suture tensioner relative to the tensioner housing to transition the suture tensioner among a plurality of tensioner configurations defined between and comprising a released configuration, in which the posts are positioned to exert no force on the suture, and a fully tensioned configuration, in which the posts are positioned to engage the suture to yield a maximum tension in the suture when the suture is connected to the implantable device, and wherein the rotator comprises an indicator configured to provide a visual indication of the tensioner configuration.

20. The suture lock assembly of claim 13, further comprising a gasket that is configured to form an at least substantially fluid-tight seal to restrict leakage out of the quick release dock port when the quick release suture mechanism is in the unlocked configuration.
